(19) United States
(12) Patent Application Publication
(10) Pub. No.: US 2008/0206770 A1
(43) Pub. Date: Aug. 28, 2008

(54) SCREENING METHODS USED TO IDENTIFY COMPOUNDS THAT MODULATE SKIN STROMAL CELLS (FIBROBLASTS) ABILITY TO MODIFY FUNCTION OF EXTRACELLULAR MATRIX

(76) Inventors: Rita Zobel, Tallinn (EE); Anzelika Liik, Tallinn (EE); Helle Sadam, Tallinn (EE); Toomas Neuman, Tallinn (EE)

Correspondence Address: BIOTECH BEACH LAW GROUP , PC, 625 BROASWAY, Suite 1210, SAN DIEGO, CA, 92101, US

(21) Appl. No.: 12/018,041

(22) Filed: Jan. 22, 2008

Related U.S. Application Data
(60) Provisional application No. 60/897,086, filed on Jan. 24, 2007.

(51) Int. Cl. C12Q 1/68; C12Q 1/37; C12Q 1/02; C12Q 1/26

(52) U.S. Cl. 435/6; 435/24; 435/29; 435/25

(57) ABSTRACT

The cellular response to cosmetic products has been characterized on the molecular level through the use of gene and protein expression technologies. Nucleic acid and protein molecules, the expression of which are induced or repressed in response to exposure to cosmetics, are identified according to a temporal pattern of altered expression post exposure. Methods are disclosed that utilized these cosmetics-regulated molecules as markers for effectiveness of cosmetics. Other screening methods of the invention are designed for the identification of compounds that modulate the response of a cell to exposure to cosmetics. The invention also provides compositions useful for drug screening or pharmaceutical purposes.

### CROSS REFERENCE TO RELATED APPLICATIONS

The present application claims benefit of priority to U.S. patent application Ser. No. 60/897,086 filed on Jan. 24, 2007 and is herein incorporated by reference in its entirety.

### TECHNICAL FIELD

The present invention relates to methods of screening for compounds with therapeutic utility and more specifically methods of identifying compounds that modulate the activity of stromal cells in the skin.

### BACKGROUND OF THE INVENTION

Wrinkles and “old” skin can have a profound impact on one's self-esteem. Indeed, the stigma attached to looking old is evidenced by the fact that Americans and Europeans spend more than $20 billion each year on cosmetics to camouflage the signs of aging. As a person ages, the skin undergoes significant changes: 1) The cells divide more slowly, and the inner layer of skin (the dermis) starts to thin. In addition, the ability of the skin to repair itself diminishes with age; and 2) The network of fibrillar proteins also known as extracellular matrix (ECM) which provides scaffolding for the surface skin layers, loosens and unravels. Skin aging is a result of unbalanced synthesis and degradation of skin matrix.

Anti-aging and anti-wrinkle skin care products are developed for one purpose—to help skin stay young, smooth and wrinkle free. Elasticity and smoothness of the skin depend in large extent on the structure of skin matrix. Skin matrix is a dynamic structure that is renewed continuously whereas its homeostasis is tightly controlled by main cellular signaling pathways. Products of more than 500 genes are directly involved in the regulation of ECM. These genes and gene products form a complicated regulatory system that has many steady states and regulatory loops. Numerous scientific papers from different laboratories clearly demonstrate that this complicated system has variations that are specific for individuals. Skin cells of each individual have specific unique characteristics including patterns of genetic polymorphisms, mutations, gene expression pattern, response to a variety of treatments, proliferation rate and many others.

Patrick Brown's laboratory was first to turn attention on individual variations of gene expression patterns of fibroblasts (Chang et al., 2002, Chang et al., 2004). His and others work has clearly shown that fibroblasts from different individuals posses unique gene expression pattern and response to variety of stimulations.

The extracellular matrix (ECM) includes an underlying network of elastin and collagen fibers and provides scaffolding for the surface skin layers. It also contains proteoglycans, numerous fibrillar proteins (fibrillins, fibulins), metalloproteinases, enzymes such as lysyl oxidase and many other minor components. Majority of active ingredients of anti-aging and anti-wrinkle skin care products stimulate synthesis of components of extracellular matrix such as different types of collagens, elastin and proteoglycans. Collagen is a structural protein found in the ECM. Collagens and proteins with collagen-like domains form large superfamilies, and the numbers of known family members are increasing constantly. Vertebrates have at least 27 collagen types with 42 distinct polypeptide chains, >20 additional proteins with collagen-like domains and 20 isoenzymes of various collagen-modifying enzymes (Myllyharju and Kivirikko. 2004). The level of individual collagen molecules in ECM is controlled by synthesis and degradation. Synthesis of different collagen molecules is largely controlled by transcriptional mechanisms. Transcription factors SMAD, SP, ETS STAT and several others mediate the effect of variety of signaling systems at the transcriptional level. Cytokines, interleukins and TGFbeta are the signaling molecules that control homeostasis of dermal ECM and mediate effect of immune and humoral systems.

Elastin fibers function together with collagen in the ECM and provide elasticity to the system. Elastic fibers are essential extracellular matrix macromolecules comprising an elastin core surrounded by a mantle of fibrillin-rich microfibrils (Kielty, Sherratt and Shuttleworth. 2002. Elastic fibers. Journal of Cell Science 115, 2817-2828). Elastin gene expression is regulated by several extracellular effectors such as IL-1b, bFGF, IGF-1, TNF-alpha and TGF-beta that initiate complex intracellular responses. At the transcriptional level these pathways modify activity of NF-kB, C/EBP. FRA, SMAD, SP and AP-1 transcription factors.

It is relatively well documented and accepted by customers that anti-aging and anti-wrinkle skin care products are not very effective. However, there is always a group of individuals who respond well to particular treatment resulting in amazing rejuvenation effects of skin. Current progress in genomics and proteomics research has clearly demonstrated that at the molecular level people vary significantly and that effective treatments of variety of disease conditions require tailored treatments that are based on molecular signatures of individuals. The same is true for anti-aging and anti-wrinkle skin care products. Individual variations in skin cells (genetic makeup, UV caused alterations) have to be considered to achieve good results by using specific skin care products with different active compounds. Unfortunately, in the area of skin care and cosmetics all customers are treated equally, without considering individual genetic, biochemical and physiological variations. Almost fifty active compounds are used in different combinations in skin care products that target elderly skin and claim to rejuvenate it. Numerous novel active compounds that target various molecular mechanisms of skin matrix homeostasis are in development. The need to match skin care products and skin molecular signature leads to development of personalized cosmetics/skin care that is based on the molecular analysis of different skin cells of individual customers.

### SUMMARY OF THE INVENTION

The present invention addresses the need for compositions and methods for the identification/determination of molecular signature/characteristics of skin cells of each individual. The present invention exploits the specific response of gene and protein expression of components of extracellular matrix and regulatory molecules to specific treatments and chemical compounds. Thus it is an object of the present invention to provide methods and compositions to identify skin care products that have maximal, enhanced or preferred effects on the skin of a specific individual. It is also an object of the present invention to provide methods of screening compounds, cosmetics or thereapeutics for their effect against skin aging and wrinkling of the skin of a specific individual.

In one aspect of the present invention, a screening method for identifying a compound that modulates the response of a skin cell to cosmetic or therapeutic products exposure is provided. In one embodiment of the present invention the method includes contacting a skin cell with a compound of interest; exposing the skin cell to one or more cosmetic or therapeutic products that in the absence of the compound of interest would induce a response, wherein the response is a pattern of gene expression associated with the extracelluar matrix (ECM); measuring the levels of a plurality of RNA or protein biomolecules in the skin cell for at least one time point after cosmetic or therapeutic products exposure, wherein the RNA or protein biomolecules are associated with the ECM; and comparing the measured levels to a control or control expression profile to determine whether a change in the pattern of gene expression occurred, thereby indicating the compound modulates the response of said skin cell to cosmetic or therapeutic products exposure.

In another aspect of the present invention, a method of screening for a compound for use as a treatment of a skin condition is provided. The method includes: a) obtaining skin cells from an individual having a skin condition in need of treatment; b) measuring the presence of a plurality of biomolecules associated with the extracelluar matrix (ECM) from a first portion of the skin cells; b) exposing a second portion of the skin cells to a compound suspected of providing a desired therapeutic effect associated with the extracellular matrix (ECM); c) measuring the presence of the plurality of biomolecules in the second portion of skin cells after exposure; d) comparing the measurements obtained from the first portion and the second portion; and e) approving the compound for treatment of said skin condition if the comparison demonstrates a desired difference in the presence of said plurality of biomolecules.

In another aspect of the present invention a composition of matter is provided including a plurality of nucleic acid molecules, the expression of which is altered by exposure to cosmetic or therapeutic products. Nucleic acid molecules of the composition are selected from at least one of the following groups: (1) molecules of extracellular matric (ECM) including structural proteins and enzymes all of which relate to the altered expression of RNA molecules in a cell exposed to cosmetic or therapeutic product; and (2) regulatory molecules including transcription regulators, splicing regulators, mRNA binding proteins, different components of cellular signaling all of which relate to the altered expression of RNA molecules in a cell exposed to cosmetic or therapeutic product.

### BRIEF DESCRIPTION OF THE FIGURES

FIG. 1 is a graphical representation of the relative abundance of gene expression of COL1A1, COL2A1, elastin, proteoglycan and TIMP1 in fibroblasts following 24 hour treatment with all-trans retanoic acid, MATRIXYL, or KAPPELASTIN.

### DETAILED DESCRIPTION OF THE PREFERRED EMBODIMENTS

### I. Definitions

Aspects of the invention utilize techniques and methods common to the fields of molecular biology, cell biology and immunology. Thus the terms used in the present disclosure should be construed with their ordinary meaning in such arts, which can be identified using many peer reviewed publications. Useful laboratory references for these types of methodologies are readily available to those skilled in the art. See, for example, Molecular Cloning, A Laboratory Manual, 2nd. edition, edited by Sambrook, J., Fritsch, B. F. and Maniatis, T., (1989), Cold Spring Harbor Laboratory Press; Harlow & Lane, Antibodies: A Laboratory Manual, Cold Spring Harbor Press, Cold Spring Harbor, N.Y. (1988). Provided for the convenience of the reader are tables providing reference to Gen bank accession numbers. All sequences referenced by Gen bank accession number including the amino acid sequences encoded therefrom, which can be identified using standard genetic code tables, are herein incorporated by reference.

The terms “nucleic acid” or “nucleic acid molecule” as used herein refer to a deoxyribonucleotide or ribonucleotide polymer in either single- or double-stranded form, and unless otherwise indicated, encompasses analogs of natural nucleotides that can function in a similar manner as the naturally occurring nucleotide. Nucleic acids of particular biomolecules may be retrieved using the Gen bank accession numbers provide herein and may be isolated, purified, cloned or synthesized using any techniques known in the art.

The term “oligonucleotide” as used herein refers the subset of nucleic acids that include a single-strand of nucleotides of 12 bases plus N bases, wherein N is a whole integer from 0 to 500. They may also include non-naturally occurring nucleotide analogs, such as those which are modified to improve hybridization or to improve detection such as the chemical addition of fluorescent moieties as used in the fluorescent detection arts.

The term “cell” as used herein is meant to include skin cells (e.g., epidermal or dermal) or precursors thereof, in a culture, tissue, or organism. In specific cases, the cell is a keratinocyte or a fibroblast cell. The cell may be from a particular patient that is to undergo a medical treatment such as the application of a cosmetic or a therapeutic or may be from a non-patient, which is not to undergo a medical treatment. The term “cell” also includes cell lines.

The term “biomolecules” as used herein refers to compounds, nucleic acids and proteins that are expressed in a cell. The term “biomolecules associated with the extracellular matrix” refers to compounds, nucleic acids and proteins that are present or secreted from a skin cell and are involved in the generation or regulation of the extracellular matrix. The term “plurality of biomolecules” as used herein refers to a group of biomolecules wherein at least two biomolecules have different function or structure.

The term “a plurality of RNA molecules” as used herein is meant an RNA sample of high complexity; the term “complexity” being used here according to standard meaning of the term as established by Britten et al. (Meth. Enzymol. (1974) 29:363).

The term “altered expression” as used herein refers to an increase or decrease in the expression level, or presence, of one or more nucleic acids, preferably one or more RNA molecules. The term “altered expression” may also refer to a relative increase or decrease in the expression level, or presence, of one or more protein molecules, which may correlate to the potential success or failure of a proposed medical treatment. Also, it will be understood by one skilled in the art that an increase or decrease in an expression level may be effected by synthesis rate, ie., transcription rate for RNA and translation rate for protein, or a change in the stability of either the RNA or protein molecules affected. Thus, all of the “responses” described herein by a cell exposed to cosmetic or therapeutic products represent “altered expression” relative to nucleic acid molecules (e.g., RNAs) or proteins normally expressed in a cell that was not exposed cosmetic or therapeutic products.

The term “expression profile” as used herein refers to a collection of data or data points corresponding to the presence, absence or abundance of a plurality of biomolecules. An “expression profile” may include the absolute or relative abundance of a plurality of biomolecules in a skin cell, a skin cell population and the like. An “expression profile” may include data such as: concentration; fold increase or decrease; percent increase or decrease; or presence or absence.

The term “quantifying” when used in the context of quantifying transcription levels of a gene can refer to absolute or relative quantification. Absolute quantification may be accomplished by inclusion of known concentration(s) of one or more target nucleic acids and referencing the hybridization intensity of unknown nucleic acids with the known target nucleic acids (e.g., through generation of a standard curve). Alternatively, relative quantification can be accomplished by comparison of hybridization signals between two or more genes, or between two or more treatments, to quantify the changes in hybridization intensity and, by implication, the changes in the transcription level. A similar approach may be taken with protein quantification.

The term “compound” as used herein includes both organic molecules and inorganic molecules. The term compound also encompasses proteins, nucleic acid molecules, carbohydrates, lipids, and combinations thereof.

### II. Methods of Screening for Compounds, Cosmetics and Therapeutics that Modulate Biomolecules including RNA or Protein Associated with the Extracellular Matrix

The present invention includes methods of identifying compounds, cosmetics and therapeutics that affect the extracellular matrix (ECM). More specifically, the methods provided herein include the detection or measurement of a plurality of biomolecules associated with the ECM and thus determine whether a skin cell's exposure to a compound, cosmetic or therapeutic affects regulation of ECM associated biomolecules. Specific applications of the present technology include among others, identification of compounds for the treatment of skin conditions and analysis of products for the treatment of skin conditions.

It is one object of the present invention to provide methods of analyzing the effect of cosmetics, therapeutics and compounds on the extracellular matrix. It is another object of the present invention to provide methods that demonstrate potential changes to expression levels across many biomolecules associated with the extracellular matrix (ECM) in response to the exposure of cosmetics, therapeutics or compounds. It is another object of the present invention to provide expression profiles that predict whether a proposed skin treatment is likely to provide a beneficial effect for a patient. It is another object of the present invention to identify whether a compound can modulate the response of a skin cell to cosmetic or therapeutic products exposure. In view of the above objects, the inventors provide the following aspects and embodiments for applications involving the detection, measurement or monitoring of a plurality of biomolecules associated with extracellular matrix (ECM) in response to compounds, cosmetics and therapeutics.

In one aspect of the present invention a method of screening for a compound that modulates the response of a skin cell to cosmetic or therapeutic products exposure is provided. The method includes: a) contacting a skin cell with a compound of interest; b) exposing the skin cell to one or more cosmetic or therapeutic products that in the absence of the compound of interest would induce a response, wherein the response is a pattern of gene expression associated with the extracelluar matrix (ECM); c) measuring the levels of a plurality of RNA or protein biomolecules obtained from the skin cell for at least one time point after cosmetic or therapeutic products exposure, wherein the RNA or protein biomolecules are associated with the ECM; and d) comparing the measured levels to a control or control expression profile to determine whether a change in the pattern of gene expression occurred, thereby indicating the compound modulates the response of said skin cell to cosmetic or therapeutic products exposure. As will become apparent to one skilled in the art, compounds identified using the following methods may inhibit the expression or presence of biomolecules associated with the ECM that would otherwise be increased upon exposure to the cosmetic or therapeutic. In other embodiments, a compound identified using the provided methods will increase the expression or presence of biomolecules associated with the ECM compared to the cosmetic or therapeutic. Thus various applications of the present invention include identify compounds that provide inhibitory, synergistic or additive effects when provided in conjunction with a cosmetic or therapeutic product.

In another aspect of the present invention, a method of screening for a compound for use as a treatment of a skin condition is provided. The method includes: a) obtaining skin cells from an individual having a skin condition in need of treatment; b) measuring the presence of a plurality of biomolecules associated with the extracelluar matrix (ECM) from a first portion of the skin cells; b) exposing a second portion of the skin cells to a compound suspected of providing a desired therapeutic effect associated with the extracellular matrix (ECM); c) measuring the presence of the plurality of biomolecules in the second portion of skin cells after exposure; d) comparing the measurements obtained from the first portion and the second portion; and e) approving the compound for treatment of said skin condition if the comparison demonstrates a desired difference in the presence of said plurality of biomolecules. Thus it will be apparent to one skilled in the art that the methods of the present invention may be used to identify new treatments for skin conditions associated with the extracellular matrix.

In another aspect of the invention a composition of matter is provided including a plurality of nucleic acid molecules, the expression of which is altered by exposure to cosmetic or therapeutic products. Nucleic acid molecules of the composition are selected from at least one of the following groups: (1) molecules of extracellular matric (ECM) including structural proteins and enzymes all of which relate to the altered expression of RNA molecules in a cell exposed to cosmetic or therapeutic product; (2) regulatory molecules including transcription regulators, splicing regulators, mRNA binding proteins, different components of cellular signaling all of which relate to the altered expression of RNA molecules in a cell exposed to cosmetic or therapeutic product. Alternatively, in another embodiment, the composition comprises a plurality of nucleic acid molecules defined to be at least one, or at least two, or at least three, or at least four, or at least five, or at least six, or at least seven, or at least eight, or at least nine, or at least ten nucleic acid molecules selected from any of those in Tables 1-20 or partial sequences.

In another aspect of the present invention a method to detect exposure of a cell to cosmetic or therapeutic products is provided, including measuring the levels of a plurality of RNA molecules in the cell by expression array analysis. Expression array analysis comprises isolating RNA from the cell post cosmetic or therapeutic products exposure, creating a test expression array through nucleic acid hybridization between a labeled probe that is complementary to the RNA, and an expression array substrate, analyzing the test expression array to create a test expression array data set, and comparing the test expression array data set to a control expression array data set to identity alterations in the expression level. The levels of the plurality of RNA molecules are then analyzed to establish a response pattern of the cell, wherein exposure of the cell to cosmetic or therapeutic products is indicated by the response pattern comprising at least one selected from any of those in Tables 1-20 or any partial sequence. In this method, the cellular response is sometimes characterized by the first response occurring from about 0.5 to about two hours post-exposure to cosmetic or therapeutic products, the second response occurring from about four to about eight hours post-exposure to cosmetic or therapeutic products, and the third response occurring from about 16 to about 24 hours post-exposure to cosmetic or therapeutic products. The response comprises an increase in expression level or a decrease in expression level.

In another aspect, the present invention provides a method for detecting exposure of a cell to cosmetic or therapeutic products by screening for a response of the cell to cosmetic or therapeutic products, the response being an altered pattern of expression determined by gene expression array analysis. The method including: (1) measuring the levels of a plurality of RNA molecules in the cell for at least one time point after cosmetic or therapeutic products exposure to establish a test pattern of expression; and (2) comparing the test pattern of expression to the response of a cell to cosmetic or therapeutic products exposure. If the pattern of expression for the cell is substantially similar to the normal response of the cell to cosmetic or therapeutic products, the cell was exposed to cosmetic or therapeutic products.

In another aspect, the present invention provides a method for detecting exposure of a cell to cosmetic or therapeutic products by screening for a response of the cell to cosmetic or therapeutic products exposure, the response being an altered pattern of expression determined by gene expression array analysis. The method comprises: (1) measuring the levels of a plurality of proteins in the cell for at least one time point after cosmetic or therapeutic products exposure to establish a test pattern of expression; and (2) comparing the test pattern of expression to the response of a cell to cosmetic or therapeutic products exposure. If the pattern of expression for the cell is substantially similar to the normal response of the cell to cosmetic or therapeutic products, the cell was exposed to cosmetic or therapeutic products.

In another aspect, the invention provides a method for detecting exposure of a cell to cosmetic or therapeutic products. This method includes measuring the levels of a plurality of protein molecules in the cell for at least one time point, wherein an altered pattern of expression is established and is indicative of cosmetic or therapeutic products exposure. This pattern comprises a first response comprising an altered pattern of expression of at least one protein that is at least 90% identical to a polypeptide encoded by a polynucleotide selected from the group consisting of the secondary first response group; a second response comprising an altered pattern of expression of at least one polynucleotide selected from the group consisting of the secondary second response group; and a third response comprising an altered pattern of expression of at least one polynucleotide selected from the group consisting of the secondary third response group. In various embodiments related to creation of a protein expression profile of a cell exposed to cosmetic or therapeutic products, ELISA is used to measure the levels of the plurality of proteins expressed in the presumptively exposed cell. In one embodiment of the method thereof, the cell is contacted with the compound in vitro. In various embodiments related to screening method thereof, ELISA is used to measure the levels of the plurality of proteins in a cell in determining the response of the cell to cosmetic or therapeutic products in the presence and absence of the compound.

In another aspect, the invention provides a screening method for the detection of a compound that simulates the response of a cell to cosmetic or therapeutic products exposure, comprising: contacting the cell with the compound; measuring a level of at least one RNA molecule in the contacted cell; and determining that the level of at least one RNA molecule in the cell after exposure to the compound is substantially similar to the level of the RNA found in the cell in response to cosmetic or therapeutic products exposure, the response of the cell to cosmetic or therapeutic products exposure being characterized by altered expression of the genes selected from any of those in Tables 1-20 or any partial sequence.

In another aspect, the invention provides a screening method for the detection of a compound that simulates the response of a cell to cosmetic or therapeutic products exposure, including: contacting the cell with the compound; measuring a level of at least one protein in the contacted cell; and determining that the level of at least one protein in the cell after exposure to the compound is substantially similar to the level of the protein found in the cell in response to cosmetic or therapeutic products exposure, the response of the cell to cosmetic or therapeutic products exposure being characterized by altered expression of the protein selected or encoded from any of those in Tables 1-20 or any partial sequence.

In another aspect, the invention provides a screening method for the detection of a compound that simulates the response of a cell to cosmetic or therapeutic products exposure, comprising: contacting the cell with the compound; measuring a level of at least one protein in the contacted cell; and determining that the level of at least one protein in the cell after exposure to the compound is substantially similar to the level of the protein found in the cell in response to cosmetic or therapeutic products exposure, the response of the cell to cosmetic or therapeutic products exposure being characterized by altered expression of the protein selected or encoded by any of those in Tables 1-20 or any partial sequence.

As will become apparent to one skilled in the art, the methods of the present invention include detecting or measuring a plurality of biomolecules associated with the extracellular matrix and monitoring changes in response to compounds, cosmetics, therapeutics or a combination thereof. Detection or measurement of such biomolecules is typically performed by detection or measurement of corresponding nucleic acid sequences, polypeptide sequences or proteins within or released from skin cells. Thus the present invention provides methods of identifying various responses of the skin cell to a compound, to cosmetic or therapeutic products exposure or a combination thereof.

The present invention includes the measurement or detection of biomolecules, including RNA, cDNA polypeptides, protein, and fragments thereof. Biomolecules may be obtained and measured from any type of skin cell believed to be involved in the generation or maintenance of the extracellular matrix (ECM). Among these skin cells include, but are not limited to epidermal cells and dermal cells. Also included are keratinocytes, Langerhans' cells, melanocytes, and fibroblasts. Keratinocytes are the major cell type of the epidermis, making up about 90% of epidermal cell.

Langerhans' cells are dendritic cells abundant in epidermis, containing large granules called Birbeck granules. Upon infection of an area of skin, the local Langerhans' cells will take up and process microbial antigens to become fully-functional antigen-presenting cells. Langerhans' cells are derived from the cellular differentiation of monocytes with the marker “Gr-1” (also known as “Ly-6c/G”). The differentiation may be obtained by stimulation by colony stimulating factor-1. They are similar in morphology and function to macrophages.

Melanocytes are cells located in the bottom layer (the stratum basale) of the skin's epidermis. When ultraviolet rays penetrate the skin and damage DNA; thymidine dinucleotide (pTpT) fragments from damaged DNA will trigger melanogenesis and cause the melanocyte to produce melanosomes, which are then transferred by dendrite to the top layer of keratinocytes.

In the preferred embodiment the expression of biomolecules obtained from fibroblasts is measured or detected. Fibroblasts synthesize and maintain the extracellular matrix of many animal tissues. Fibroblasts provide a structural framework (stroma) for many tissues, and play a critical role in wound healing. They are the most common cells of connective tissue in animals. The main function of fibroblasts is to maintain the structural integrity of connective tissue by continuously secreting precursors of the extracellular matrix. Fibroblasts secrete the precursors of all the components of the extracellular matrix, primarily the ground substance and a variety of fibers. The composition of the extracellular matrix determines the physical properties of connective tissues. Fibroblasts are morphologically heterogeneous with diverse appearances depending on their location and activity. Unlike the epithelial cells lining the body structures, fibroblasts do not form flat monolayers and are not restricted by a polarizing attachment to a basal lamina on one side, although they may contribute to basal lamina components in some situations.

The methods of the present invention may be applied to the treatment of skin condition by screening for compounds, cosmetics or therapeutics that demonstrate desired activity on a cellular or in vitro level. Examples of skin conditions that may benefit from the present invention include skin that is wrinkled, loose, discolored and the like. By identifying the biomolecules associated with the particular skin condition, physicians may taylor a treatment regimen that target the cause or target rejuvenation via cellular processes.

Skin cells for use with the present invention may be primary cells obtained from a patient or may be obtained from a variety of cell lines (ATCC, Manassas Va.). Skin cells may cultured, may be identified by one or more detectable markers such as surface markers specific to the cell type or may be detected by examining the morphology. Techniques for isolating and culturing skin cells are well known in the cellular arts. Examples of cells that may be used with the present invention include any that are associated or contribute to the generation or maintenance of the extracellular matrix (ECM). Among these include dermal cells and epidermal cells. Further examples include keratinocytes, a Langerhans' cells, melanocytes and fibroblasts.

Skin cells are contacted with a compound of interest and may be exposed to cosmetics or therapeutics. As one skilled in the art recognizes, there are many ways in which a cell may be “contacted” with a compound. Contact may be in vivo or in vitro. For example, the compound may be placed in a carrier liquid medium, such as phosphate buffered saline (PBS) or tissue culture media, and when cells are incubated in this media, the compound contacts or touches the cell. Alternatively, contact may be through a cream or a gel which is applied topically to the skin. Thus cells contacted with a compound may be a cell in a culture, a cell in an isolated tissue, or a cell in an organism, such as a skin cell (epidermal or dermal). One skilled in the art will recognize that in some instances it may be desirable to split a cell population into two subpopulations or more to allow for control experiments and the like. Thus a first population may remain untreated as a control and a second population may be treated as the experimental or test population.

Compounds are tested for their ability to modulate the expression of biomolecules associated with the ECM by studying their effect on skin cells and optionally cosmetic or therapeutic products. Compounds that may be of particular interest to those practicing the present invention may be organic or inorganic compounds, nucleic acid molecules such as DNA, cDNA, RNA, iRNA, siRNA, anti-sense RNA, polypeptides, proteins, protein fragments and the like. Preferably compounds provided in the present invention are suspected of affecting at least one biological process such as inducing, activating, inhibiting, or reducing the presence of a biomolecule associated with the extracellular matrix (ECM). In another preferred embodiment the compound is suspected of inhibiting or enhancing an effect from exposure of a skin cell to a cosmetic or therapeutic product or a skin treatment.

In some embodiments of the present invention the methods identify compounds that induce or activate the expression, presence or release of biomolecules associated with the extracellular matrix. Thus compounds of the present invention may induce or activate gene transcription resulting in variations of the presence of RNA associated with the ECM or may induce or activate translation, which varies the abundance of ECM related protein. Activation may be identified by measuring the presence (or level) of one or more biomolecules associated with the ECM before and after contact with the compound. Activation or an increase is deemed to occur if an increase in the presence of the biomolecule is observed compared to a control. In various embodiments thereof, RNA may be isolated from the skin cell, reverse transcribed and then probed for the presence of cDNA that corresponds to the biomolecule of interest. The results may then be compared to a suitable control such cDNA obtained from skin cells that were not treated or a control expression profile as described in the present invention. In one further embodiment a DNA microarray is used to detect transcript associated with a biomolecule and an expression profile generated therefrom.

In other embodiments the methods identify compounds that inhibit biomolecules associated with the extracellular matrix or reduce their presence. Compounds that inhibit or reduce the presence of biomolecules may be desirable when the biomolecule's function is to degrade ECM components. As a nonlimiting example, as the skin gets older the presence of collagen and elastin tend to decrease. Since skin cells produce biomolecules such as collagenases that digest collagen, a compound that prevents such degradation would be desirable as a treatment against wrinkling or aging of the skin.

In other embodiments of the present invention, methods of identifying compounds that inhibit the skin cells' modulation of biomolecules in response to exposure of a cosmetic or therapeutic are provided. Thus the methods may be used to identify compounds that counteract exposure of a cosmetic or therapeutic. Such compounds may be used to identify or further study regulatory pathways that are stimulated in response to the presence of the cosmetic or therapeutic. Alternatively, such compounds may partially inhibit the skin cells' ability to modulate ECM associated biomolecules when the cosmetic or therapeutic provides a response that is in part desired and in part undesired. Thus when a cosmetic or therapeutic causes an adverse effect in addition to a desired effect, it may be desirable to identify a compound capable of inhibiting or reducing the adverse effect. As an example, many skin treatments include the compound retinoic acid; however in addition to its beneficial effects retinoic acid can cause redness. Thus compounds identified using methods described herein may selectively counteract the undesirable effect (redness) while not effecting other desired effects associated with retinoic acid.

In other embodiments of the present invention compounds may be identified that enhance or induce an effect when provided in combination with a cosmetic or therapeutic. The identification of such compounds may lead to the development of next generation compositions that include a combination of therapies. Thus the present invention may be used to identify compounds with synergistic or additive effects when used in combination with a cosmetic or therapeutic.

As described herein the methods of the present invention may include the exposure of a skin cell to a cosmetic or therapeutic product or an active compound contained therein. Expression analysis, such as gene expression array analysis, of biomolecules can indicate whether or to what degree the cosmetic or therapeutic affects expression of biomolecules associated with the extracellular matrix (ECM). Thus the methods of the present invention may provide expression analysis corresponding to various cosmetics or therapeutics for a potential patient or for patients in general. Any cosmetic or therapeutic treatment may be provided that is suspected of affecting the ECM. Such cosmetic or therapeutic treatments include those offered in the skin treatment industry and may include those provide in creams, lotions, sprays and the like.

As described throughout the present invention a plurality of biomolecules are detected or measured for their presence, absence or abundance, whether absolute or relative. Thus the monitoring of biomolecules permits analysis of a compound's or product's effect on the cell and thus the ECM. Biomolecules are typically obtained directly from skin cells using techniques described herein or known in the art such as RNA isolation or protein techniques. Many such techniques are described within the present document and are known in the art, with the caveat that the present invention seeks to identify biomolecules associated with the ECM. Among the techniques that may be adapted for use with the present invention include microarray hybridization, electrophoresis, capillary electrophoresis, liquid chromatography, reverse transcription polymerase chain reaction (RT-PCR), enzyme-linked immunosorbent assay (ELISA), Western Blot Northern Blot and the like. In the preferred embodiment gene array expression analysis adapted with a plurality of biomolecules provided or derived from Tables 1-22, such as complementary or partial sequences. As used throughout the specification, the measurement of RNA is intended to include the measurement of cDNA that is reverse transcribed from RNA. Further information regarding specific techniques to detect or measure biomolecules are provided below. Such techniques include the measurement of biomolecules including RNA, cDNA, polypeptides and protein. The disclosure of which is herein incorporated by reference in its entirety.

The number of biomolecules detected or measured is intended to be nonlimiting. As a nonlimiting example, Table 22 summarizes the analysis of about 36 biomolecules associated with all trans retinoic acid treatment of human dermal fibroblasts. Analysis was conducted on the RNA and protein level. Although nonlimiting, in one embodiment the modulation or expression of at least two biomolecules is provided. In another embodiment the modulation or expression of at least three biomolecules is provided. In another embodiment the modulation or expression of at least four biomolecules is provided. In another embodiment the modulation or expression of at least five biomolecules is provided. In another embodiment the modulation or expression of at least six biomolecules is provided. In another embodiment the modulation or expression of 7-10 biomolecules is provided. In another embodiment the modulation or expression of 10-15 biomolecules is provided. In another embodiment the modulation or expression of 15-20 biomolecules is provided. In another embodiment the modulation or expression of 20-30 biomolecules is provided. In another embodiment the modulation or expression of 30-40 biomolecules is provided. In another embodiment the modulation or expression of 40-50 biomolecules is provided. In another embodiment the modulation or expression of 50-75 biomolecules is provided. In another embodiment the modulation or expression of 75-100 biomolecules is provided. In further embodiments, the expression of greater than 100, 500 or 1000 biomolecules are provided. Thus the present invention is not intended to be limited with respect to the number of biomolecules so long as the biomolecules exist or are later identified.

Measurement or detection of biomolecules, such as the measurement of gene expression, may occur at a variety of time points. In one embodiment measurement of biomolecules associated with the ECM occurs at a single time point, which is after exposure of the skin cell to a compound, cosmetic or therapeutic. In this embodiment the expression of the plurality of biomolecules in the treated sample may be compared to a control profile. In other embodiments, measurement or detection of a plurality of biomolecules occurs before and after such exposure or contact. Measurements before and after exposure or contact may be preferred when the effect of a compound, cosmetic or therapeutic is to be compared to the cells of the same individual before such exposure or contact. In further embodiments, measurements or detection occurs over time.

Other embodiments of the method thereof vary according to the time period post-cosmetic or therapeutic products exposure defining the first response, second response, and third response. In one embodiment, the first response is from about 0.5 hours to about two hours post-exposure to cosmetic or therapeutic products. In another embodiment, the second response is from about four hours to about eight hours post-exposure to cosmetic or therapeutic products. In another embodiment, the third response is from about 16 hours to about 24 hours post-exposure to cosmetic or therapeutic products. In other embodiments measurements across time points such as every 2, 3, 4, 5 or 6 hours may occur such as over 24 to 48 hours.

Levels of biomolecules associated with the ECM may be evaluated to determine whether an increase or decrease in the presence or expression of one or more of the plurality of biomolecules occurs. In one embodiment the level of RNA or protein after exposure to a cosmetic or therapeutic product is compared to the level of RNA or protein prior to such exposure. In another embodiment, the level of RNA or protein is compared over a time line or over multiple time points. In another embodiment the level of RNA or protein after exposure is compared to a control profile. Thus the comparisons may be between any desired time point, such as before or after contacting a cell with a compound of interest; or before or after exposing a cell to a cosmetic or therapeutic product. In addition it is not meant as a requirement that all levels are checked at each and every time point.

The data obtained using the present methods may be incorporated into a profile that summarizes all or some measurements. Thus profiles such as an expression profile may be generated for each cosmetic or therapeutic product or may be generated according to class, predicted effect and the like. In other embodiments profiles are be generated as controls that correspond to particular cosmetic or therapeutic products or may represent patient data. Thus profiles may be used to predict outcome, compare treatment groups or track progression of modulation. Profiles may include relative amounts, absolute amounts, percent increase or decrease over measurements, fold increase or decrease over a series of measurements and the like.

The generation of expression profiles or profiles in response to compounds, cosmetics or therapeutics may allow a physician to identify a particular treatment by correlating a treatment with current status. Thus by evaluating the levels of a variety of compounds, the physician is able to choose a treatment that specifically targets the patient's cellular condition. For example, a patient showing decreased collagen may require the administration of a treatment that increases collagen production or prevents collagenase activity. Thus treatments may be tayored to desired outcome, which may include decreasing biomolecules that degrade the ECM and increasing biomolecules that build or replenish the ECM.

In further embodiments, a patient's cells are cultured and tested across a panel of potential treatments. By screening those cultures that respond positively, the physician is capable of identifying treatments shown to work in culture, thus eliminating many potential treatments that were not effective in culture.

As will be apparent to one skilled in the art levels of biomolecules such as RNA or protein may be compared to controls or other samples to identify whether modulation has occurred and to what degree. Comparisons of biomolecule levels may be between cells obtained from the same individual or source, different individuals or sources, different or the same treatment groups and the like. Thus by comparing levels of biomolecules such as through gene expression analysis and/or protein analysis, the effects of compounds, cosmetic and therapeutics may be determined. These effects may show increases, decreases, no effect and the like.

### III. Biomolecules Associated with the Extracellular Matrix and Their Analysis

The present invention includes measuring the presence of a plurality of biomolecules associated with the extracellular matrix (ECM) to identify, suggest or confirm whether exposure to a cosmetic or therapeutic will have a desired effect on the ECM. In other embodiments, the methods of the present invention measure the presence of a plurality of biomolecules associated with the ECM to determine the effect of a compound on the ECM with or without a cosmetic or therapeutic. Measurements to assess the effect of compounds, cosmetics or therapeutics on the ECM are performed using skin cells, which are associated with the ECM. More specifically levels of a plurality of biomolecules are measured from skin cell cultures or populations after exposure to cosmetics or therapeutics to determine whether altered expression in biomolecule expression occurs. Naturally the levels of biomolecules may also be measured before such exposure for comparison. Preferably, biomolecules are measured at the RNA, polypeptide or protein level. Thus RNA or protein obtained from skin cells are obtained and measured using techniques known in the molecular biology, biochemistry and cellular biology arts. Such shifts in such biomolecule expression allow the identification, suggestion or confirmation that exposure induces an effect on the EMC.

An expression profile may be formed from the measured levels of the plurality of biomolecules. The expression profile may be compared to a suitable control or to levels measured from additional samples, such as samples obtained from the same individual or different individual. Examples of biomolecules that may be considered when generating an expression profile for use with the present invention include any believed to be associated with the extracellular matrix (ECM), including but not limited to collagen, fibrillin, elastin, fibronectin, a proteoglycan, an enzyme, a matrix metallopeptidase (MMP), a metalloproteinase inhibitor (TIMP). Furthermore, each of the above biomolecules may represent groups of biomolecules, which may be further subdivided into subgroups of biomolecules.

In some embodiments, one or more collagen proteins or RNAs (or cDNAs obtained from reverse transcription) are measured or detected. Collagen is a structural protein found in the ECM. Many cosmetics and therapeutics claim to increase collagen levels. Thus the present invention may determine whether the particular compound, cosmetic or therapeutic will increase the production of collagen in skin cells and whether such claims would be accurate for a particular individual and thus whether treatment would be appropriate. However since collagen may be referred to as a group, family or superfamily of many members having structural similarities, the present invention may seek to identify which biomolecules within the collagen family are affected by such treatment. Examples of collagens that may be of particular interest include COL1A1, COL1A2, COL2A1, COL3A1, COL4A1, COL4A2, COL4A3, COL4A4, COL5A1, COL5A2, and COL7A1. These collagens may be detected or measured by any of the methods provided for detecting or measuring RNA or protein referred to in the present invention, and may include hybridizing a nucleic acid sample to a nucleic acid sequence obtained from any of the collagen sequences. Moreover the inventors of the present invention provide primers for PCR amplification of each of the above listed collagens in Table 21 and the size of the amplified fragment, which are also encompassed within methods of the present invention. The nucleic acid and polypeptide sequences which are included within collagen biomolecules may be found in various databases including those referred to in the present invention such as Gen bank, specific accession numbers are provided in the tables.

In some embodiments, one or more fibrillin polypeptides or RNA are measured or detected. Fibrillin is a glycoprotein that is essential for the formation of elastic fibers found in connective tissue. Fibrillin is one of the components found in the extracellular matrix and thus may be used to assess or predict the effectiveness of a therapeutic compound for the treatment of skin conditions associated with the ECM. Among the fibrillins included in the present invention include Fibrillin-1 and Fibrillin-2. Fibrillin-1 is a major component of the microfibrils that form a sheath surrounding elastin. Fibrillin-2 is believed to play a role in early elastogenesis.

In some embodiments of the present invention, one or more proteoglycan proteins, polypeptides or nucleic acid sequences such as DNA, cDNA and RNA encoding a proteoglycan are detected or measured. Proteoglycans are a special class of glycoproteins that are heavily glycosylated. They include a core protein with one or more covalently attached glycosiminoglycan chain(s). These glycosaminoglycan (GAG) chains are long, linear carbohydrate polymers that are negatively charged under physiological conditions, due to the occurrence of sulfate and uronic acid groups. Proteoglycans are a major component of the animal extracellular matrix. They form large complexes, both to other proteoglycans, and to fibrous matrix proteins (such as collagen). In addition, proteoglycans are believed to be involved in binding cations (such as sodium, potassium and calcium) and water, and also regulating the movement of molecules through the ECM. Proteoglycans are also believed to affect the activity and stability of proteins and signaling biomolecules within the ECM. Among the proteoglycans that may be measured or detected for screening compounds useful as cosmetics or therapeutic compounds for skin conditions associated with the extracellular matrix include, but are not limited to aggrecan and the small leucine rich repeat proteoglycans (SLRP) decorin, fibromodulin, and lumican.

In some embodiments of the present invention, one or more enzyme proteins, polypeptides or nucleic acid sequences such as DNA, cDNA and RNA encoding an enzyme associated with the ECM or regulation of ECM are detected or measured. A variety of enzymes are present in skin cells that may affect the presence or absence of biomolecules with the ECM. Among these include Lysyl oxidase. Lysyl oxidase is a copper-dependent amine oxidase that plays a critical role in the biogenesis of connective tissue matrices by crosslinking the extracellular matrix proteins, collagen and elastin. Levels of Lysyl oxidase increase in many fibrotic diseases, while expression of the enzyme is decreased in certain diseases involving impaired copper metabolism. Thus when treating a fibrotic disease it may be preferred to reduce or screen for an effect that reduces Lysyl oxidase, but when treating a condition where Lysyl oxidase is reduced such as where insufficient crosslinking of ECM biomolecules occurs it may be preferred to screen for effects that increase the presence or activity of Lysyl oxidase.

In some embodiments of the present invention one or more matrix metallopeptidases (MMP) biomolecules including nucleic acid sequences such as DNA, cDNA and RNA are detected or measured or polypeptides resulting therefrom. MMPs are zinc-dependent endopeptidases. The MMPs share a common domain structure including the pro-peptide, the catalytic domain and the haemopexin-like C-terminal domain, which is linked to the catalytic domain by a flexible hinge region. MMPs degrade the extracellular matrix (ECM). Thus it may be desirable to identify compounds or cosmetics that decrease the presence or activity of MMPs or increase inhibitors thereof. Among MMPs of interest may include, but are not limited to MMP2 (gelatenase-A), MMP3 (stromelysin 1), MMP7 (matrilysin), MMP9 (gelatenase-B), MMP-11 (stromelysin 3), MMP12 (macrophage metalloelastase), MMP13 (collagenase 3), MMP19 (RSA 1 or stromelysin-4) and MMP20 (Enamyelysin).

In some embodiments of the present invention a TIMP biomolecule such as a DNA, cDNA, RNA, polypeptide, protein or fragment thereof is detected or measured. TIMPs are matrix metalloprotease (MMP) inhibitors. TIMPs are known as inhibitors of MMPs and are believed to have an anti-apoptoitic function. Transcription of TIMPs are highly inducible in response to many cytokines and hormones and thus treatment with such cytokines or hormones may be a proposed therapy if TIMPs are abnormally present. Within the TIMP family, TIMP1, TIMP2, TIMP3 and TIMP4 may be of particular use with the present invention.

The descriptions of the biomolecules and there usefulness obtaining a profile associated with the state or condition of an extracellular matrix is non-limiting. Thus additional targets believed to be associated with the extracellular matrix (ECM) are also included. Tables 1-20 below provide a variety of biomolecules that may be measured to determine the state of the ECM and affects thereon by the exposure to a cosmetic, therapeutic or compound. Thus the contents of the below table may be used to identify sequences such as nucleic acid sequences, polypeptide sequences, partial sequences or fragments thereof to design assays for detection or measurement of biomolecules associated with the ECM for screening, including for the generation of complementary nucleic acid sequences used for the preparation of probes for hybridization to the nucleic acid sequences. Thus the citations provided below are intended to refer and fully incorporate the particular sequence data and the descriptions of the biomolecules provided therein. The citations are intended to herein incorporate by reference in their entirety the sequence data referred to as well as all descriptions of the sequence data or biomolecules themselves as if fully provided below and in a sequence listing. Tables 1-8 are intended to have the headings of Gen bank #, UniGene, Gene ID and Protein as referred to in Table 1. Thus tables 1-8 are intended to be broken up for the ease of one skilled in the art to identify biomolecules encompassed within the present invention and are not intended to be limiting.

TABLE 1
Collagens

| Biomolecule | Gen bank # | UniGene | Gene ID | Protein |
|---|---|---|---|---|
| COL1A1 | NM_000088 | Hs.172928 | 1277 | NP_000079.1 |
| COL1A2 | NM_000089 | Hs.489142 | 1278 | NP_000080 |
| COL2A1, | NM_001844 | Hs.408182 | 1280 | NP_001835.2 |
| transcr.var.1 | | | | |
| COL3A1 | NM_000090 | Hs.443625 | 1281 | NP_000081.1 |
| COL4A1 | NM_001845 | Hs.17441 | 1282 | NP_001836.1 |
| COL4A2 | NM_001846 | Hs.508716 | 1284 | NP_001837.1 |
| COL4A3 v1-v6 | NM_000091 | Hs.471525 | 1285 | NP_000082.2 |
| COL4A4 | NM_000092 | Hs.418040 | 1286 | NP_000083.2 |
| COL5A1 | NM_000093 | Hs.210283 | 1289 | NP_000084.2 |
| COL5A2 | NM_000393 | Hs.445827 | 1290 | NP_000384.1 |
| COL7A1 | NM_000094 | | | |

TABLE 2
Elastins

| | | | | |
|---|---|---|---|---|
| Elastin ELN | NM_000501 | Hs.252418 | 2006 | NP_000492.1 |

TABLE 3
Fibronectins

| | | | | |
|---|---|---|---|---|
| FN 1 transcr.v.1 | NM_212482 | Hs.203717 | 2335 | NP_997647.1 |

TABLE 4
Fibrillins

| | | | | |
|---|---|---|---|---|
| Fibrillin-1 (FBN1) | NM_000138 | Hs.146447 | 2200 | NP_000129.2 |
| Fibrillin-2 (FBN2) | NM_001999 | Hs.519294 | 2201 | NP_001990.2 |

TABLE 5
Proteoglycans

| | | | | |
|---|---|---|---|---|
| Lumican (LUM) | NM_002345 | Hs.406475 | 4060 | NP_002336.1 |
| decorin v.A1 (DCN) | NM_001920 | Hs.156316 | 1634 | NP_001911.1 |
| fibromodulin (FMOD) | NM_002023 | Hs.519168 | 2331 | NP_002014.2 |
| aggrecan | NM_001135 | | 176 | NP_001126 |

TABLE 6
Enzymes

| | | | | |
|---|---|---|---|---|
| Lysyl oxidase (LOX) | NM_002317 | Hs.102267 | 4015 | NP_002308.2 |

TABLE 7
MMPs

| | | | | |
|---|---|---|---|---|
| MMP2 | NM_004530 | Hs.513617 | 4313 | NP_004521.1 |
| MMP3 | NM_002422 | Hs.375129 | 4314 | NP_002413.1 |
| MMP7 | NM_002423 | Hs.2256 | 4316 | NP_002414.1 |
| MMP9 | NM_004994 | Hs.297413 | 4318 | NP_004985.2 |
| MMP10, | NM_002425 | Hs.2258 | 4319 | NP_002416.1 |
| stromelysin 2 | | | | |
| MMP-11, | NM_005940 | Hs.143751 | 4320 | NP_005931.2 |
| stromelysin 3 | | | | |
| MMP12 (56) | NM_002426 | Hs.1695 | 4321 | NP_002417.2 |
| MMP13 | NM_002427 | Hs.2936 | 4322 | NP_002418.1 |
| MMP19 | NM_002429 | Hs.154057 | 4327 | NP_002420.1 |
| MMP20 | NM_004771 | Hs.302383 | 9313 | NP_004762.2 |

TABLE 8
Metalloproteinase Inhibitors (TIMP)

| | | | | |
|---|---|---|---|---|
| TIMP1 | NM_003254 | Hs.522632 | 7076 | NP_003245.1 |
| TIMP2 | NM_003255 | Hs.104839 | 7077 | NP_003246.1 |
| TIMP3 | NM_000362 | Hs.297324 | 7078 | NP_000353.1 |
| TIMP4 | NM_003256 | Hs.567349 | 7079 | NP_003247.1 |

Tables 9-12 include biomolecules that may be detected or measured within the scope of the present invention. Thus expression profiles or profiles used to compare biomolecules may include the below provided biomolecules. Encompassed within the present invention are the names and listings referenced included DNA, cDNA, RNA and polypeptide sequences transcribed or translated therefrom using the genetic code and amino acid tables well known in the molecular biology arts. The citations are provided for the convenience of the reader. Thus the Gen bank sequences are fully incorporated herein by reference in their entirety as if the sequences were provided in the sequence listing provided herein and herewith, including their corresponding RNA and polypeptide sequences transcribed and translated therefrom.

TABLE 9
Basal Complex

| Gene | Gen Bank # |
|---|---|
| TBP | NM_003194 |
| TBPL1 | NM_004865 |

TABLE 10
GTF Transcription factors

| Gene | Gen Bank # |
|---|---|
| GTF2A1 v.1 | NM_015859 |
| GTF2A1 v.2 | NM_201595 |
| ALF v.1 TFIIA-alpha/beta-like factor | NM_006872 |
| ALF v.2 | NM_172196 |
| GTF2B | NM_001514 |
| GTF2E1 | NM_005513 |
| GTF2E2 | NM_002095 |
| GTF2F1 | NM_002096 |
| GTF2F2 | NM_004128 |
| GTF2H1 | NM_005316 |
| GTF2H2 | NM_001515 |
| GTF2H3 | NM_001516 |
| GTF2H4 | NM_001517 |
| GTF2H5 | NM_207118 |
| XPD/ERRC2 | NM_000400 |
| XPB/ERCC3 | NM_000122 |
| cdk7 | NM_001799 |
| cyclin H/CCNH | NM_001239 |
| MNAT1 | NM_002431 |
| GTF2I v.1 | NM_032999 |
| GTF2I v.2 | NM_033000 |
| GTF2I v.3 | NM_033001 |
| GTF2I v.4 | NM_001518 |

TABLE 11
TAFII family TFIID

| Gene | Gen Bank # |
|---|---|
| TAF1 v.1 (TAFII250) | NM_004606 |
| TAF1 v.2 (TAFII250) | NM_138923 |
| TAF2(TAFII150) | NM_003184 |
| TAF3 (TAFII140) | XM_291729 |
| TAF4 (TAFII 130/135) | NM_003185 |
| TAF4b (TAFII105) | XM_290809 |
| TAF5 (TAFII100) | NM_139052 |
| TAF5L (PAF65b) | NM_014409 |
| TAF6 v.1 (TAFII80) | NM_005641 |
| TAF6 v.2 | NM_139315 |
| TAF6 v.3 | NM_139122 |
| TAF6 v.4 | NM_139123 |
| TAF6L (PAF65a) | NM_006473 |
| TAF7 (TAFII55) | NM_005642 |
| TAF7L (TAF2Q) | NM_024885 |
| TAF8 (TAFII43/TBN) | NM_138572 |
| TAF9 v.1 (TAFII32/31) | NM_003187 |
| TAF9 v.2 | NM_016283 |
| TAF9 v.3 | NM_001015891 |
| TAF9 v.4 | NM_001015892 |
| TAF9L (TAFII31L | NM_015975 |
| TAF10 (TAFII30) | NM_006284 |
| TAF11 (TAFII28) | NM_005643 |
| TAF12 (TAFII20/15) | NM_005644 |
| TAF13 (TAFII18) | NM_005645 |
| TAF15 v.1 (TAFII68) | NM_139215 |
| TAF15 v.2 | NM_003487 |

TABLE 12
B-TFIID

| Gene | Gen Bank # |
|---|---|
| BTAF1 RNA polymerase II | NM_003972 |
| DR1 (down-regulator of transcription 1) | NM_001938 |
| DRAP1 DR1-associated protein 1 | NM_006442 |

TABLE 13
Mediator Complex TRAP/DRIP/ARC/CRSP

| Gene | Gen Bank # |
|---|---|
| MED1 | NM_004774 |
| MED4 | NM_014166 |
| MED6 | NM_005466 |
| MED7 | NM_004270 |
| MED8 | NM_052877 |
| MED11 | NM_001001683 |
| MED14 | NM_004229 |
| MED15 | NM_001003891 |
| MED17 | NM_004268 |
| MED18 | NM_017638 |
| MED19 | NM_153450 |
| MED20 | BC012618 |
| MED21 | NM_004264 |
| MED22 | NM_133640 |
| MED26 | NM_004831 |
| MED27 | NM_004269 |
| MED30 | NM_080651 |
| MED31 | NM_016060 |

TABLE 14
Tail Module

| Gene | Gen Bank # |
|---|---|
| MED16 | NM_005481 |
| MED23 | NM_004830 |
| MED24 | NM_014815 |
| MED25 | NM_030973 |

TABLE 15
Not present in PC2

| Gene | Gen Bank # |
|---|---|
| MED12 | NM_005120 |
| MED13* | NM_005121 |
| SRB10 | BC069634 |
| SRB11 | NM_005190 |
| *MED13L | NM_015335 |

TABLE 16
Chromatin Remodeling Complexes,
BAF Complex (SWI/SNF like)

| Gene | Gen Bank # |
|---|---|
| SMARCA1 | NM_003069 |
| SMARCA2 | NM_003070 |
| SMARCA3 | NM_003071 |
| SMARCA4 (hSNF2B) | NM_003072 |
| SMARCA5 | NM_003601 |
| SMARCB1 | NM_003073 |
| SMARCC1 (BAF155) | NM_003074 |
| SMARCC2 v.1 (BAF170) | NM_003075 |
| SMARCC2 v.2 | NM_139067 |
| SMARCD1 | NM_003076 |
| SAMRCD2 | NM_003077 |
| SMARCD3 | NM_003078 |
| SMARCE1 (BAF57) | NM_003079 |
| SMARCE1r (BRAF35/25) | NM_006339 |
| BAF53 | NM_178042 |
| SRCAP | NM_006662 |
| ATRX | NM_000489 |
| SWI1 (ARID1B) | NM_017519 |

TABLE 17
hINO80 complexAAA+ ATPasees, SWR1 homologs

| Gene | Gen Bank # |
|---|---|
| RUVBL1 | NM_003707 |
| RUVBL2 | NM_006666 |
| CHD1 | NM_001270 |
| CHD2 | NM_001271 |
| CHD3 v1 | NM_001005273 |
| CHD3 v2 | NM_005852 |
| CHD3 v3 | NM_001005271 |

TABLE 18
Co-activators

| Gene | Gen Bank # |
|---|---|
| NCOA1 | NM_147233; |
| | NM_003743 |
| | NM_147223 |
| NCOA2 | NM_006540 |
| NCOA3 | NM_181659 |
| | NM_006534 |
| NCOA4 | NM_005437 |
| NCOA5 | NM_020967 |
| NCOA6 | NM_014071 |
| NCOA7 | NM_181782 |
| PC4 | NM_006713 |
| TRRAP | NM_003496 |
| PPARGC1A | NM_013261 |
| NRIP1 | NM_003489 |
| CITED1 | NM_004143 |
| CITED2 | NM_006079 |
| CITED4 | NM_133467 |
| CARM1 | NM_199141 |
| TMF1 | NM_007114 |
| TGFB1I1 (Hic-5/ARA55) | NM_001042454 |
| ASCC1 | NM_015947 |
| ASCC2 | NM_032204 |
| ASSC3 | NM_006828 |
| PRPF6 | NM_012469 |
| SNW1 | NM_012245 |
| RBM14 | NM_006328 |

TABLE 19
Co-repressors

| Gene | Gen Bank # |
|---|---|
| NCOR1 | NM_006311 |
| NCOR2 | NM_006312 |
| CtBP | NM_001328 |
| RCOR1 REST corepressor 1 | NM_015156 |
| BCOR BCL6 co-repressor | NM_017745 |
| PELP1 | NM_014389 |
| MTA3 | NM_020744 |
| TRIM28 | NM_005762 |

TABLE 20
Factors

| Biomolecule | Gen bank # |
|---|---|
| SR-A1 | NM_021228 |
| SF1 | NM_004630 |
| SF4 | NM_172231 |
| SFRS1 | NM_006924 |
| SFRS2 | NM_003016 |
| SFRS3 | NM_003017 |
| SFRS4 | NM_005626 |
| SFRS5 | NM_006925 |
| SFRS6 | NM_006275 |
| SFRS9 | NM_003769 |
| SFRS10 | NM_004593 |
| SFRS12 | NM_139168 |
| SFRS14 | AF518874 |
| FUSIP1 | NM_006625 |
| | NM_054016 |
| SFRS2IP | NM_004719 |
| SRPK1 | NM_003137 |
| SRP46 | NM_032102 |
| SR protein rA4 | XM_047889 |
| SRm160 | AF048977 |
| RNPS1 | NM_006711 |
| SRrp35 | NM_080743 |
| SRp-55 | U30883 |
| SRp55 | U30828 |
| SRp55 | U30829 |
| SRp75 | L14076 |
| U1-70K | NM_003089 |
| U2AF1RS1 | NM_005083 |
| U2AF1RS2 | NM_005089 |
| U2AF1 | NM_006758 |
| U2AF65 | NM_007279 |
| ZNF265 | NM_005455 |
| SCNM1 | |
| PRPF3 | NM_004698. |
| PRPF4 | NM_004697 |
| PRPF8 | NM_006445. |
| PRPF31 | NM_015629 |
| PRP17/CDC40 | NM_015891 |
| PTBP1 | NM_002819 |
| hnRNP A1 | NM_031157 |
| hnRNP A2 | |
| hnRNP B1 | |
| hnRNP C1 | |
| hnRNP C2 | |
| hnRNP K | NM_002140 |
| SF3B1 | NM_012433 |
| SF3B2 | NM_006842 |

TABLE 21
RNA binding factors

| Factor | Gen bank # |
|---|---|
| Nova | |
| Marlin-1 | AY382340 |
| IMP-1 | NM_006546 |
| IMP-2 | NM_006548 |
| IMP-3 | NM_006547 |
| HuB | |
| HuD | |
| HuC | |
| HuR | |
| SFPQ | NM_005066 |
| NONO | NM_007363 |
| TINO | AF458084 |
| RKHD1 | NM_203304 |

ECM Associated Microarrays

The biomolecules provided in Tables 1-20 may be used in the generation of expression arrays and the like for the measurement of gene expression within a sample. Techniques utilized include those known in the molecular biology, biochemistry and cellular biology arts. In preferred embodiments nucleic acid sequences are provided in a DNA microarray format. A DNA microarray (also commonly known as gene or genome chip, DNA chip, or gene array) is a collection of microscopic DNA spots, commonly representing single genes, arrayed on a solid surface by covalent attachment to a chemical matrix. Microarray technology evolved from Southern blotting, whereby fragmented DNA is attached to a substrate and then probed with a known gene or fragment. DNA microarrays can be used to detect DNA (e.g., in comparative genomic hybridization); it also permits detection of RNA (most commonly as cDNA after reverse transcription) that may or may not be translated into proteins, which is referred to as “expression analysis” or expression profiling. DNA arrays are different from other types of microarray only in that they either measure DNA or use DNA as part of its detection system. Qualitative or quantitative measurements with DNA microarrays utilize the selective nature of DNA-DNA or DNA-RNA hybridization under high-stringency conditions and frequently utilize fluorophore-based detection such as fluorescent labeled probes. DNA arrays are commonly used for expression profiling, i.e., monitoring expression levels of thousands of genes simultaneously, or for comparative genomic hybridization. Thus a DNA microarray incorporating nucleic acid sequences associated with the ECM such as those selected from the group of nucleic acid sequences in any of tables 1-20 or partial sequences thereof, can be used to monitor expression and monitor changes in expression in response to exposure with cosmetics, therapeutics or compounds.

As will be apparent, different embodiments of the invention are directed to low and high density nucleic acid and oligonucleotide arrays. Depending on the intended application, arrays may be constructed using oligonucleotides, cDNAs, genomic clones, etc.; such a determination is well within the knowledge of one skilled in the art. Typically, oligonucleotide arrays are utilized in a high density format. Genomic clones, cDNAs and other polynucleotides greater than about 500 base pairs are easily utilized in a low density setting, but to obtain a high density array with these polynucleotides it is most easily accomplished by robotic application to a substrate. U.S. Pat. No. 5,143,854 and PCT Patent Publication Nos. WO 90/15070 and WO 92/10092 teach the use of light-directed combinatorial synthesis of high density oligonucleotide arrays, and the synthesis of high density arrays is also described in U.S. Pat. Nos. 5,744,305, 5,800,992 and 5,445,934, each of which herein incorporated by reference in their entirety.

Those skilled in art will recognize that arrays of DNA can either be spatially arranged, as in the commonly known gene or genome chip, DNA chip, or gene array, or can be specific DNA sequences tagged or labeled such that they can be independently identified in solution. The traditional solid-phase array is a collection of microscopic DNA spots attached to a solid surface, such as glass, plastic or silicon chip. The affixed DNA segments are known as probes (although some sources will use different nomenclature such as reporters), thousands of which can be placed in known locations on a single DNA microarray.

Many methods for immobilizing nucleic acids on a variety of substrates are known in the art. A wide variety of organic and inorganic polymers, as well as other materials, both natural and synthetic, can be employed as the material for the solid surface of a gene array. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials which may be employed include, but are not limited to, paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

Microarrays can be manufactured in different ways, depending on the number of probes under examination, costs, customization requirements, and the type of scientific question being asked. Arrays may have as few as 10 probes to up to 390,000 micron-scale probes. Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays.

In spotted microarrays, the probes are oligonucleotides, cDNA or small fragments of PCR products that correspond to mRNAs. The probes are synthesized prior to deposition on the array surface and are then “spotted” onto glass. A common approach utilizes an array of fine pins or needles controlled by a robotic arm that is dipped into wells containing DNA probes and then depositing each probe at designated locations on the array surface. The resulting “grid” of probes is ready to receive complementary cDNA or cRNA “targets” derived from experimental or clinical samples.

In oligonucleotide microarrays, the probes are short sequences designed to match parts of the sequence of known or predicted open reading frames. Oligonucleotide arrays can be produced by printing short oligonucleotide sequences designed to represent a single gene or family of gene splice-variants by synthesizing the desired sequence directly onto the array surface instead of depositing intact sequences.

One common technique used to produce oligonucleotide arrays include photolithographic synthesis on a silica substrate where light and light-sensitive masking agents are used to “build” a sequence one nucleotide at a time across the entire array. Thus each applicable probe is selectively “unmasked” prior to bathing the array in a solution of a single nucleotide, then a masking reaction takes place and the next set of probes are unmasked in preparation for a different nucleotide exposure. After many repetitions, the sequences of every probe become fully constructed. However techniques including maskless array synthesis may also be used with the present invention.

Microarray systems known in the art may be adapted for use with the present invention by incorporating nucleic acid sequences corresponding to a plurality of biomolecules associated with the ECM. Thus the nucleic acid sequences used in the array may include partial sequences, genomic clones, cDNAs, oligonucleotides and the like corresponding to biomolecules associated with the ECM. A listing of biomolecules and their corresponding Gen bank accession number is provided as Tables 1-20. Thus one skilled in the art may prepare an ECM expression array by identifying the biomolecules of interest from Tables 1-20, accessing the nucleic acid sequence from Gen bank using the cited Gen bank accession numbers, and constructing an array using array construction techniques in conjunction with the nucleic acid sequences identical, complementary or at least 90% identical or complementary to sequences obtained from the Gen bank accession number of partial sequences therewithin.

The present invention is intended to encompass sequences having homology to those referred to by Gen bank accession number provide in Tables 1-20 or partial sequences within. Thus the nucleotide sequences used in the construction of an array for expression analysis of biomolecules associated with the ECM includes but is not limited to partial sequences (less than full sequences cited by Gen bank accession number) and sequences that are 90%, 91%, 92%, 93%, 94%, 95%, 96%, 97%, 98%, 99% or 100% homologous to nucleic sequences accessible by the cited Gen bank accession numbers including partial sequences. Sequences that hybridize to nucleic acid sequences provide in the Gen bank accession numbers under highly stringent conditions (as provided in Sambrook et al) may also be used. Thus one skilled in the art can choose the length of the desired probe and its homology by accessing nucleic acid sequences referred to by Gen bank accession number.

In a particularly preferred embodiment, the composition of matter comprises nucleic acid molecules that are nucleotides selected to have a length of 12 bases plus N bases, wherein N is a whole integer from 0 to 500. In one embodiment, the biomolecule nucleic acid probes are long, such as 500 or more nucleotides in length. In another embodiment the biomolecule nucleic acid is 100-500 nucleotides in length. In another embodiment of the present invention the biomolecule nucleic acid is 60-100 nucleotides in length. In another embodiment the biomolecule nucleic acid probes are from about 25-60 nucleotides in length. In another embodiment the biomolecule nucleotide probes are from about 20-25 nucleotides in length. In another embodiment, the biomolecule nucleotide probes are small, such as from about 12-20 nucleotides in length. In another embodiment, oligonucleotides that are 21 bases in length are provided. In the most preferred embodiment a composition including the nucleic acid sequences corresponding to biomolecules associated with ECM is characterized as a gene array. Longer probes may be more specific to individual target genes; whereas shorter probes may be spotted in higher density across the array and are cheaper to manufacture.

One skilled in the art would recognize that hybridization of nucleic acid sequences is dependent on homology. Determining homology may include determining the percentage of sequence identity. The “percentage of sequence identity” or “sequence identity” may be determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g., nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT is calculated using default gap weights.

Optimal alignment of sequences for comparison may be conducted by the local homology algorithm of Smith and Waterman (Adv. Appl Math. (1981) 2:482); by the homology alignment algorithm of Needleman and Wunsch (J. Mol. Biol. (1970) 48:443); by the search for similarity method of Pearson and Lipman (Proc. Natl. Acad. Sci. (USA) (1988) 85:2444); by computerized implementations of these algorithms (including, but not limited to, CLUSTAL in the PC/Gene program by Intelligenetics (Mountain View, Calif.) GAP, BESTFIT, FASTA, and TFASTA in the Wisconsin Genetics Software Package, Genetics Computer Group (GCG) (Madison, Wis.); or by inspection. In particular, methods for aligning sequences using the CLUSTAL program are well described by Higgins and Sharp (Gene (1988) 73:237 244), (CABIOS (1989) 5:151 153), and both the BLAST and the PSI-BLAST programs (Altschul, et al (1997), Nucleic Acids Res. 25:3389 3402).

Measurement or Detection of Nucleic Acid Biomolecules Associated with Extracellular Matrix (ECM)

The level of an RNA molecule or a plurality of RNA molecules may be measured by any means known in the art. Methods of detecting and/or quantifying the transcript(s) of one or more gene(s) of this invention (e.g., mRNA or cDNA made therefrom) using nucleic acid hybridization techniques are known to those of skill in the art.

Preferably, the screening method measures the levels of the plurality of RNAs by an expression array analysis. This analysis includes isolating RNA from the cell for at least one time point post-cosmetic or therapeutic products exposure, creating a test expression array through nucleic acid hybridization between the labeled probe that is complementary to the isolated RNA and an expression array substrate, and analyzing the test expression array to create a test expression array data set. The test and control data sets are then compared to identify a modulation of the response of the cell exposed to cosmetic or therapeutic products. The modulation indicates that the compound modulates the response of a cell exposed to cosmetic or therapeutic products.

In addition methods for evaluating the presence, absence, or quantity of gene reverse-transcribed cDNA involves a Southern blot transfer and subsequent quantitation using nucleic acid hybridization technology. Alternatively, in a Northern blot, mRNA is directly quantitated. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target mRNA.

The probes used herein for detection of the gene(s) of this invention can be full length or less than the full length of the gene. Shorter probes are empirically tested for specificity. Preferably, nucleic acid probes are 20 bases or longer in length. (See, Sambrook et al., supra, for methods of selecting nucleic acid probe sequences for use in nucleic acid hybridization). Visualization of the hybridized portions allows the qualitative determination of the presence or absence of gene(s) of this invention.

Measurement or Detection of Protein Biomolecules Associated with the Extracellular Matrix

Methods of the present invention may include the detection of one or more proteins associated with the extracellular matrix (ECM). Thus in some embodiments the methods include detecting the presence or absence or measuring the amount, whether absolute or relative, of at least one protein in response to exposure or contact of a cell with a compound, cosmetic or therapeutic. The methods may include measuring the levels of a plurality of protein molecules in the cell for at least one time point, and comparing the levels to a pattern of expression, wherein the pattern of expression is established and the pattern is indicative of cosmetic or therapeutic products exposure.

Preferably, the levels of a plurality of protein molecules are measured by enzyme-linked immunosorbent assays (ELISAs). In other embodiments of the invention, the polypeptides encoded by the gene sequences identified by the invention as cosmetic or therapeutic product regulated may be detected and quantified by any of a number of methods well known to those skilled in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), immunofluorescent assays, Western blotting, and the like.

As known by those skilled in the art, an ELISA assay (e.g., Coligan, et al. (1991) Curr. Protocols Immunol. 1(2):Chapter 6) includes preparing an antibody (preferably a monoclonal antibody) specific to a target protein. To the reporter antibody is attached a detectable reagent such as radioactive isotope, fluorescent tag or enzyme (e.g., horseradish peroxidase). A sample is removed from a host and incubated on a solid support (e.g. a polystyrene dish) that binds the proteins in the sample. Any free protein binding sites on the dish are then covered by incubating with a non-specific protein like bovine serum albumen. Next, the monoclonal antibodies attach to any target proteins attached to the polystyrene dish. All unbound monoclonal antibody is washed away with buffer. The reporter antibody is then placed in the dish resulting in binding of the reporter antibody to any monoclonal antibody bound to the target protein. Unattached reporter antibody is then washed out of the dish. The detectable reagent is then detected to identify the protein of interest bound by the antibody specific for the target protein. For example, when horseradish peroxidase is used as a detectable label, peroxidase substrate is added to the dish, and the amount of color developed in a given time period is a measurement of the amount of target protein present in a given volume of sample when compared against a standard curve.

Other assays useful for the measurement of protein levels include: radioimmunoassays, competitive-binding assays, Western blot analysis, and “sandwich” assays. In one representative sandwich assay, the target protein is passed over a solid support and binds to target-specific antibody attached to the solid support. A second antibody is then bound to the target protein. A third antibody which is labeled and specific to the second antibody is then passed over the solid support and binds to the second antibody and an amount target protein can then be indirectly quantified. In a competition assay, antibodies specific to the target protein are attached to a solid support and labeled target protein and a sample derived from the host are passed over the solid support. The amount of label detected, for example, by liquid scintillation chromatography can be correlated to a quantity of target protein in the sample.

Skin Cells from Different Individuals Respond Differently to Cosmetic Compounds and Drugs—Basis for Personalized Cosmetics and Skin Care.

Effect of skin care products and drugs varies between individuals and this variation depends on the biological response of skin cells to active compounds in clinical or cosmetic products. Smoothness and strength of the skin depends mostly on the quality of the ECM, meaning how well it is assembled and does it contain functionally and structurally important components in correct ratios. On the other hand homeostasis of the ECM depends on the synthesis and degradation of its components whereas degradation is controlled mostly by extracellular proteinases such as matrix metallo proteinases (MMPs). Activity of MMPs is controlled by tissue inhibitors of MMPs (TIMPs). To summarize, ECM structure and function depends on the balance of matrix fibrillary proteins, MMPs and TIMPs.

Analysis of the synthesis of fibrillary proteins (both mRNA and protein level) and degradation following treatment of skin cells with drugs and skin care products will enable dermatologists and cosmetics to evaluate effect of individual compounds on the biological response of skin cells. Based on the response of cells they can recommend treatments that have the most beneficial effects on person skin.

### EXAMPLES

### Example 1

### Identification of Gene and Protein Sequences Regulated by Exposure to All-Trans Retinoic Acid

### Methods

### Cell Culture and Treatment

Human dermal fibroblasts were grown in DMEM cell culture media containing 10% fetal bovine serum and 1% penicillin/streptomycin (Invitrogen). For all experiments, third-passage fibroblasts were used one day after reaching confluence. Cells were treated with all-trans retinoic acid (atRA, 10-7 M) for 24 hours. After 24 hour treatment RNA and protein was isolated and analyzed for the expression of components of ECM and transcriptional regulators.

### RNA Preparation

Total RNA from various human tissues was purified using RNAwiz (Ambion, USA) and subjected to subsequent DNase I treatment using the DNA-Free kit (Ambion). Total RNA from cancer cells was purified by using 4PCRmini kit (Ambion). First-strand cDNAs were synthesized with Superscript III reverse transcriptase (Invitrogen, USA) and 5 μg of total RNA using oligo d(T) priming in a final reaction volume of 50 μL

### RT-PCR Analysis

The cDNA was amplified by PCR using the 2.5 U Hot-Firepol (Solis BioDyne, Estonia) and buffer Yellow (Naxo, Estonia). Reaction was performed in a 10 ul volume, using 0.5 ul cDNA as a template. Following primer sets (Table 21) were designed for this study:

TABLE 21

Primers for RT-PCR Analysis and Corresponding Size
of Amplified Fragments

| | Fragment | SEQ | | |
|---|---|---|---|---|
| Molecule | Size | ID NO. | Sense/Antisense | Primer Sequence |
| COL1A1 | 206 | 1 | Sense | GCCGTGACCTCAAGATGTG |
| | | 2 | Antisense | GCCGAACCAGACATGCCTC |
| COL1A2 | 125 | 3 | Sense | AATTGGAGCTGTTGGTAACGC |
| | | 4 | Antisense | CACCAGTAAGGCCGTTTGC |
| COL2A1 | 270 | 5 | Sense | ACACTGGGACTGTCCTCTG |
| | | 6 | Antisense | GTCCAGGGGCACCTTTTTCA |
| COL3A1 | 226 | 7 | Sense | AGGTCCTGCGGGTAACACT |
| | | 8 | Antisense | ACTTTCACCCTTGACACCCTG |
| COL4A1 | 201 | 9 | Sense | AGGGGTCGGAGAGAAAGGTG |
| | | 10 | Antisense | GGTCCTGTGCCTATAACAATTCC |
| COL4A2 | 217 | 11 | Sense | GAGCCTGGATTGGTCGGTTTC |
| | | 12 | Antisense | GGTGGAGGGTGTCTGATGG |
| COL4A3 | 202 | 13 | Sense | CCATAGCCGTTCACAGCCAAA |
| | | 14 | Antisense | TAGTTGCACGTTCCTCTTCCA |
| COL4A4 | 240 | 15 | Sense | ATAAGGGTCCAACTGGTGTTCC |
| | | 16 | Antisense | CTCCCTGAATACCTTTAACGGC |
| COL5A1 | 106 | 17 | Sense | TCAACCTGTCAGATGGCAAGT |
| | | 18 | Antisense | CGGTCGAGGAATTTGGTGG |
| COL5A2 | 181 | 19 | Sense | ACCACAGGGTTTACAAGGACA |
| | | 20 | Antisense | TCCTGCAAATCCCACTTCACC |
| COL7A1 | 194 | 21 | Sense | GCCAACCTCCGACTTCTTCTT |
| | | 22 | Antisense | CTGTCCACTGTACTCTCAAGGA |
| Elastin ELN | 199 | 23 | Sense | CGCCCAGTTTGGGTTAGTTC |
| | | 24 | Antisense | CACCTTGGCAGCGGATTTTG |
| FN 1 | 203 | 25 | Sense | CCCCATTCCAGGACACTTCTG |
| | | 26 | Antisense | GCCCACGGTAACAACCTCTT |
| Fibrillin-1 | 183 | 27 | Sense | CAGGACAGGCCCATGTTTTAC |
| | | 28 | Antisense | CCCGTGCGGATATTTGGAATG |
| Fibrillin-2 | 240 | 29 | Sense | GTGCATTGTCCCGATTTGTAGA |
| | | 30 | Antisense | CGTCCACCATTCTGACATCCAT |
| Fibulin-2 | | | | |
| FBLN2 | 217 | 31 | Sense | TGTCCTGTGAAGACATCAACG |
| | | 32 | Antisense | GAGCCCTTGGTGTTCTGGC |
| FBLN5 | 91 | 33 | Sense | GCCCTACTCGAACCCCTACT |
| | | 34 | Antisense | GGAGATCGTGGGATAGTTTGGA |
| Lumican | 185 | 35 | Sense | TTTCAATGTGTCATCCCTGGTTG |
| | | 36 | Antisense | CCAAACGCAAATGCTTGATCTT |
| decorin | 189 | 37 | Sense | GTCCTGGAGCATTTACACCTTT |
| | | 38 | Antisense | TGGTGCCCAGTTCTATGACAA |
| fibromodulin | 132 | 39 | Sense | ACTTCCTCACGGCCATGTACT |
| | | 40 | Antisense | TGGCATTGTCAAAGACGCCT |
| aggrecan | 98 | 41 | Sense | CTGCTTCCGAGGCATTTCAG |
| | | 42 | Antisense | CTTGGGTCACGATCCACTCC |
| Lysyl | | 43 | Sense | CGACCCTTACAACCCCTACAA |
| oxidase | 232 | 44 | Antisense | GGCCAGACAGTTTTCCTCCG |
| MMP2 | 119 | 45 | Sense | CTTCCAAGTCTGGAGCGATGT |
| | | 46 | Antisense | TACCGTCAAAGGGGTATCCAT |
| MMP3 | 206 | 47 | Sense | CGGTTCCGCCTGTCTCAAG |
| | | 48 | Antisense | CGCCAAAAGTGCCTGTCTTTA |
| MMP7 | 118 | 49 | Sense | GGAGGAGATGCTCACTTCGAT |
| | | 50 | Antisense | AGGAATGTCCCATACCCAAAGA |
| MMP9 | 230 | 51 | Sense | CATTTCGACGATGACGAGTTGT |
| | | 52 | Antisense | CGGGTGTAGAGTCTCTCGC |
| MMP-11 | 152 | 53 | Sense | TCTACACCTTTCGCTACCCAC |
| | | 54 | Antisense | CTCCAGCGGTGCAATCTCATT |
| MMP12 | 192 | 55 | Sense | CTCTTCCCCTGAACAGCTCTA |
| | | 56 | Antisense | CAGTTGCCCGGTCACTTT |
| MMP13 | 61 | 57 | Sense | TTTCAACGGACCCATACAGTTTG |
| | | 58 | Antisense | CATGACGCGAACAATACGGTTA |
| MMP19 | 125 | 59 | Sense | CCGTGGACTACCTGTCACAAT |
| | | 60 | Antisense | TGAGACTGGAAGTTCAGATGCT |
| MMP20 | 134 | 61 | Sense | GAGTTCTGTCGAGGTGGACAA |
| | | 62 | Antisense | CCCCGTGATCTCCATTTTCAA |
| TIMP1 | 121 | 63 | Sense | GGGTTCCAAGCCTTAGGGG |
| | | 64 | Antisense | TTCCAGCAATGAGAAACTCCTC |
| TIMP2 | 136 | 65 | Sense | AAGCGGTCAGTGAGAAGGAAG |
| | | 66 | Antisense | GGGGCCGTGTAGATAAACTCTAT |
| TIMP3 | 120 | 67 | Sense | TGCAACTTCGTGGAGAGGTG |
| | | 68 | Antisense | CACAAAGCAAGGCAGGTAGTA |
| TIMP4 | 125 | 69 | Sense | CTGCTACACAGTACCCTGTACC |
| | | 70 | Antisense | TGCCGTCAACATGCTTCATAC |

The thermal cycling protocols were as follows: initial denaturation of template DNA and heat-activation of polymerase (15 min at 95° C.) followed by 40 cycles denaturation (30 sec at 95° C.) annealing (58° C.) and extension (90 sec). PCR products were analysed on a 1% agarose gel. For sequencing, PCR reactions were purified using PCR purification kit (Qiagen, USA) and subcloned into TOPO TA cloning vector. Fragments were sequenced at GATC Biotech sequencing services (Germany).

### Western Blot Analysis

Cells were lysed in Cell Disruption Buffer supplied with the Paris kit (Ambion) and protein concentration was determined using Bradford reagent (Pierce). Proteins were separated on 10% SDS-PAAG and transferred into the PVDF membrane (Biorad). Filters were incubated overnight using antibodies recognizing MED16 (sc-5363 or sc-5365, Santa Cruz, USA) diluted 1:200 in 5% non-fat dry milk. Signal was detected using ECL reagent (Pierce).

### Results

Treatment of fibroblasts with atRNA results in altered expression of genes that are related to ECM homeostasis. Since different derivatives of retinoic acid are used in numerous cosmetic products then it is important to know how retinoic acid affects expression of genes that encode for ECM components. These data also provide information concerning specific pathways of induction or repression of components of ECM. Following table shows the effect of atRA on expression of mRNA and protein of different ECM components.

TABLE 22
Effect of all trans retinoic acid on synthesis of components
of ECM. mRNA levels were analyzed using RT-PCR and protein levels
were analyzed using Western blot. Numbers show fold induction
(+) or suppression(−) compared to control level.

| | mRNA | protein |
|---|---|---|
| Biomolecule | level | level |
| COL1A1 | 2 | 2 |
| COL1A2 | 4 | 2 |
| COL2A1 | 5 | 3 |
| COL3A1 | 2 | 2 |
| COL4A1 | 1 | 1 |
| COL4A2 | 3 | 1 |
| COL4A3 | 2 | 1 |
| COL4A4 | 1 | 1 |
| COL5A1 | (−)2 | 1 |
| COL5A2 | (−)3 | (−2) |
| COL7A1 | 2 | |
| Elastin ELN | 4 | 2 |
| FN 1 | 1 | |
| Fibrillin-1 | 1 | |
| Fibrillin-2 | 1 | |
| Fibulin-2 | 3 | |
| FBLN2 | (−)3 | |
| FBLN5 | 2 | |
| Lumican | 1 | |
| decorin | 6 | 4 |
| fibromodulin | 1 | |
| aggrecan | 1 | |
| Lysyl oxidase | 8 | 3 |
| MMP2 | 2 | 2 |
| MMP3 | (−)7 | (−)3 |
| MMP7 | 2 | |
| MMP9 | 2 | |
| MMP-11 | 7 | 3 |
| MMP12 | 2 | |
| MMP13 | 5 | |
| MMP19 | 1 | |
| MMP20 | 1 | |
| TIMP1 | 2 | |
| TIMP2 | (−)4 | |
| TIMP3 | 3 | |
| TIMP4 | 1 | |

Our results clearly demonstrate that retinoids affect homeostasis of dermal ECM produced by fibroblasts in vitro. These results also show that similar analysis can be used to validate cosmetic compounds and it gives a good platform for high throughput screening of drug candidates.

### Example 2

### Fibroblasts Isolated from Different Individuals Respond Differently to Treatments with Variety of Bioactive Molecules

### Methods

### Cell Culture and Treatment

Human dermal fibroblasts were grown in DMEM cell culture media containing 10% fetal bovine serum and 1% penicillin/streptomycin (Invitrogen). For all experiments, third-passage fibroblasts were used one day after reaching confluence. Cells were treated with all-trans retinoic acid (atRA, 10-7 M), Matrixyl (1 μM) and KappaElastin (1 μM) for 24 hours. After 24 hour treatment RNA and protein was isolated and analyzed for the expression of components of ECM and transcriptional regulators.

### RNA Preparation

Total RNA from various human tissues was purified using RNAwiz (Ambion, USA) and subjected to subsequent DNase I treatment using the DNA-Free kit (Ambion). Total RNA from cancer cells was purified by using 4PCRmini kit (Ambion). First-strand cDNAs were synthesized with Superscript III reverse transcriptase (Invitrogen, USA) and 5 μg of total RNA using oligo d(T) priming in a final reaction volume of 50 μL

### RT-PCR Analysis

The cDNA was amplified by PCR using the 2.5 U Hot-Firepol (Solis BioDyne, Estonia) and buffer Yellow (Naxo, Estonia). Reaction was performed in a 10 ul volume, using 0.5 ul cDNA as a template. Following primer sets were designed for this study:

TABLE 23
Primers for RT-PCR Analysis

| | | | Frag |
|---|---|---|---|
| Molecule | SEQ ID: sense | SEQ ID: antisense | size |
| COL1A1 | SEQ ID NO: 1 | SEQ ID NO: 2 | 206 |
| COL2A1 | SEQ ID NO: 3 | SEQ ID NO: 4 | 270 |
| Elastin ELN | SEQ ID NO: 23 | SEQ ID NO: 24 | 199 |
| decorin | SEQ ID NO: 37 | SEQ ID NO: 38 | 189 |
| TIMP1 | SEQ ID NO: 63 | SEQ ID NO: 64 | 121 |

### Results

Majority of active ingredients of anti aging and anti wrinkle skin care products stimulate synthesis of components of extracellular matrix such as different types of collagens, elastin and proteoglycans. We analyzed effect of all trans retinoic acid and peptides palmitoys pentapeptide-3 (Matrixyl, Pal-KTTKS), kappa elastin (elastin peptides) on synthesis of ECM components using skin fibroblasts from 30 individuals. Retinoic acid, Matrixyl and KappaElastin are advertised as equally good active ingredients of skin care anti aging and anti wrinkle products having somewhere 20-50% effect in different advertised studies.

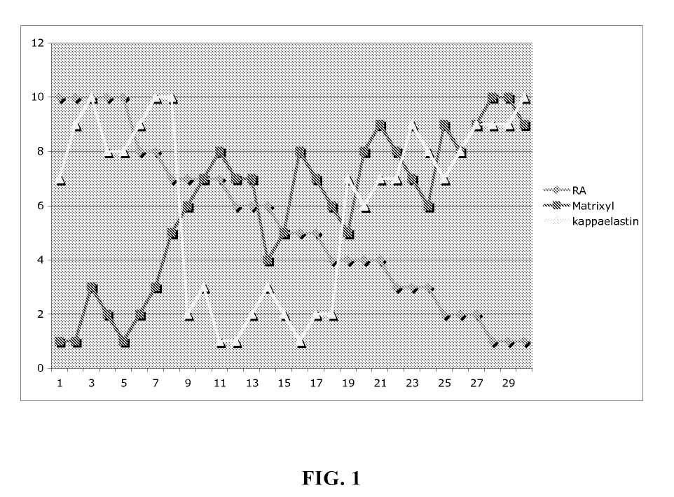

Referring to FIG. 1, we analyzed synthesis of COL1A1, COL2A1, elastin, proteoglycan and TIMP1 gene expression levels following 24 hour treatment of fibroblasts with all-trans retinoic acid, MATRIXYL and KAPPAELASTIN. The combined score of expression stimulation of 5 genes was calculated and blotted for each fibroblast isolate. FIG. 1 Presented analysis clearly demonstrates the presence of 3 groups of fibroblasts that have different response to retinoids, Matrixyl and KappaElastin. These data clearly show that skin fibroblasts isolated from different individuals with specific genetic makeup respond differently to treatments that can also explain why some people respond well and others do not respond well to variety of skin care products. This biological variation provides a good biological rational to use molecular diagnostics for matching cosmetics product and skin cells of specific individual (personalized cosmetics).

### Example 3

### Alteration of Expression of Components of ECM in Respons Eto TGFbeta and atRA Treatments Varies in Fibroblasts Isolated from Different Individuals

### Methods:

### Cell Culture

Fibroblasts were isolated from 17 adult individuals and cultured for gene expression analysis in standard conditions with no treatment or in the presence of all-trans retinoic acid (1 μM) and TGFβ1 (10 ng/ml) at 37° C. for 24 hours.

### RNA Preparation

Total RNA was extracted from cells using RNA aqueous kit (Ambion, USA). First-strand cDNAs were synthesized with Superscript III reverse transcriptase (Invitrogen, USA) and 1 μg of total RNA using oligo d(T) priming in a final reaction volume of 40 μL.

### Real Time RT-PCR Analysis

Levels of specific mRNAs were measured by real-time RT-PCR using Platinum SYBR Green qPCR SuperMix UDG (Invitrogen) according the protocol on the LightCycler 2.0 (Roche, US). The cycling program steps were: for one cycle 50° C. 2 min, for one cycle 95° C. 2 min and for 45 cycles: 95° C. 10 s, 60° C. 10 s, 72° C. 10 s. All samples were run in triplicates. Results were analyzed with the comparative Ct method (from Applied Biosystems manual; Livak and Schmittgen, 2001). Every sample was normalized for the housekeeping gene GAPDH. Transcript levels in the atRA and TGFb1 treated samples were compared to control samples.

Table 24. Effect of atRA and TGFβ1 on the expression of components of ECM, nuclear hormone receptors and coregulators varies in fibroblasts isolated from different individuals.

TABLE 24

| | | Individual variation of | | Individual variation of gene | |
|---|---|---|---|---|---|
| | | gene expression in RA | | expression in TGFbeta1 | |
| Gene | Gene bank I [symbol] | treatment | | treatment | |
| Collagens | | | | | |
| COL1A1 | NM_000088 | 9.5 | (from −6.25 to 3.25) | 8.86 | (from −1.11 to 7.75) |
| COL1A2 | NM_000089 | 4.96 | (from −3.13 to 1.83) | 8.44 | (from 1.14 to 9.58) |
| COL3A1 | NM_000090 | 5.17 | (from −2.28 to 2.89) | 8.57 | (from −1.75 to 6.82) |
| COL4A1 | NM_001845 | 6.15 | (from −3.97 to 2.18) | 13.7 | (from 2.52 to 16.22) |
| COL4A4 | NM_000092 | 1.63 | (from −2.72 to −1.09) | — | |
| COL5A1 | NM_000093 | 7.15 | (from −4.76 to 2.39) | 7.69 | (from −1.61 to 6.08) |
| COL5A2 | NM_000393 | 4.35 | (from −1.41 to 2.94) | 2.12 | (from 1.3 to 3.42) |
| Elastin | | | | | |
| ELN | NM_000501 | 5.46 | (from −2.44 to 3.02) | 13.96 | (from −3.33 to 10.63) |
| Fibronectin | | | | | |
| FN 1 transcr.v.1 | NM_212482 | 4.77 | (from −3.63 to 1.14) | 6.98 | (from −3.23 to 3.75) |
| Metalloproteinases | | | | | |
| MMP1, collagenase 1 | NM_002421 | 7.09 | (from −3.38 to 3.71) | 7.02 | (from −5.05 to 1.97) |
| MMP2 gelatinase | NM_004530 | 5.05 | (from −2.7 to 2.35) | 4.77 | (from −2.51 to 2.26) |
| MMP3 stromelysin | NM_002422 | 6.4 | (from −3.73 to 2.67) | 5.8 | (from −3.22 to 2.58) |
| MMP7 | NM_002423 | 16.46 | (from −14.77 to 1.69) | 32.8 | (from −29.96 to 2.84) |
| MMP10, | NM_002425 | 12 | (from −5.15 to 6.84) | 45.93 | (from −5.0 to 40.93) |
| stromelysin 2 | | | | | |
| MMP19 | NM_002429 | 3.41 | (from −2.4 to 1.01) | — | |
| Metalloproteinase | | | | | |
| inhibitor TIMP | | | | | |
| TIMP1 | NM_003254 | 4.21 | (from −1.09 to 3.12) | 5.73 | (from −2.45 to 3.28) |
| Nuclear receptors | | | | | |
| RARA | NM_000964 | 4.44 | (from −1.53 to 2.91) | — | |
| RARG | NM_000966 | 3.37 | (from −1.45 to 1.92) | — | |
| RXRA | NM_002957 | 4.34 | (from −1.75 to 2.59) | — | |
| RXRG | NM_006917 | 4.01 | (from −2.02 to 1.99) | — | |
| PPARG | NM_138712 | 4.59 | (from −1.64 to 2.95) | 17.71 | (from −16.51 to 1.2) |
| NR coregulators | | | | | |
| NCOA1 | NM_147233 | 5.68 | (from −2.99 to 2.69) | 5.64 | (from −2.76 to 2.88) |
| NCOA2 | NM_006540 | 5.63 | (from −1.95 to 3.68) | 1.58 | (from −2.54 to −0.96) |
| NCOA3 | NM_181659 | 6.16 | (from −2.02 to 4.14) | 6.21 | (from −4.81 to 1.4) |
| NCOA5 | NM_020967 | 3.1 | (from −1.25 to 1.85) | 4.56 | (from −3.13 to 1.43) |
| NCOA6 | NM_014071 | 3.43 | (from −1.44 to 1.99) | 2.82 | (from 1.64 to 1.18) |
| NCOA7 | NM_181782 | 4.44 | (from −1.85 to 2.59) | — | |
| PC4 | NM_006713 | 3.57 | (from −1.8 to 1.77) | — | |
| NCOR1 | NM_006311 | 4.2 | (from −2.23 to 1.97) | 7.31 | (from −2.54 to 4.77) |
| CBP | NM_004380 | 5.32 | (from −2.15 to 3.17) | 4.53 | (from −2.24 to 2.29) |
| CTGF | NM_001901 | 5.04 | (from −2.4 to 2.64) | 12.8 | (from −1.8 to 11.00) |

[symbol]

indicates data missing or illegible when filed

### Results

We analyzed effect of atRA on gene expression of different collagens, MMPs, TIMP and transcriptional co-regulators in dermal fibroblasts isolated from 17 individuals using real time PCR technique. Analyses results showed that response of individual isolates of fibroblasts had significant and even opposite effect of retinoic acid on many analyzed genes (Table). In different fibroblast isolates retinoic acid induced or repressed expression of different genes up to 8-fold (Col1A1, Col4A1, Elastin, MMP1, MMP7) (see Table 24).

To identify mechanisms that cause the opposite effect of retinoic acid in different fibroblast we analyzed expression of nuclear hormone receptors (RARs, RXRs, PPARγ) and their different co-regulators in cells treated with atRA. Real time RT-PCR results show that effect of atRA on expression of nuclear hormone receptors shows less variation between different individuals than its effect on expression of different co-regulators (Table 24). Expression of coactivators NCOA1, NCOA2 and NCOA3 in response to atRA has highest variation between individual fibroblasts.

Effect of TGFβ1 on gene expression of different ECM genes was noticeably stronger than effect of atRA, which is expected since TGFβ1 is a well-characterized profibrotic factor (Table 24). Gene expression of majority of collagens is strongly upregulated and there is also high individual variation (up to 13-fold) between individual fibroblasts. Opposite to expression of collagen genes TGFβ1 suppresses significantly expression of metalloproteinases and PPARγ. Expression of PPARγ that has antifibrotic effect (Lakatos, et al., 2007) varies strongly between different individuals (up to 17 fold).

Those skilled in the art will recognize, or be able to ascertain, using no more than routine experimentation, many equivalents to the specific embodiments of the invention described herein. Such equivalents are intended to be encompassed by the following claims.

What is claimed is:

1. A screening method for identifying a compound that modulates the response of a skin cell to cosmetic or therapeutic products exposure, comprising:
a) contacting a skin cell with a compound of interest;
b) exposing said skin cell to one or more cosmetic or therapeutic products that in the absence of said compound of interest would induce a response, wherein said response is a pattern of gene expression associated with the extracelluar matrix (ECM);
c) measuring the levels of a plurality of RNA or protein biomolecules in said skin cell for at least one time point after cosmetic or therapeutic products exposure, wherein said RNA or protein biomolecules are associated with the ECM; and
d) comparing the measured levels to a control or control expression profile to determine whether a change in said pattern of gene expression occurred, thereby indicating the compound modulates the response of said skin cell to cosmetic or therapeutic products exposure.

2. The screening method according to claim 1, wherein said skin cell is selected from the group consisting of a keratinocyte, a Langerhans cell, a melanocyte, and a fibroblast.

3. The screening method according to claim 1, wherein said levels are measured by array expression analysis or ELISA.

4. The screening method according to claim 1, wherein said at least one time point is measured from about one (1) hour to about ninety-six (96) hours after exposure.

5. The screening method according to claim 1, wherein said biomolecules are selected from the group consisting of a collagen, a fibrillin, an elastin, a fibronectin, a proteoglycan, an enzyme, a matrix metallopeptidase (MMP), a metalloproteinase inhibitor (TIMP), and a combination thereof.

6. The screening method according to claim 5, wherein:
a) said collagen is selected from the group consisting of COL1A1, COL1A2, COL2A1, COL3A1, COL4A1, COL4A2, COL4A3, COL4A4, COL5A1, COL5A2, and COL7A1; or
b) said fibrillin is Fibrillin-1 or Fibrillin-2; or
c) said proteoglycan is selected from the group consisting of aggrecan, decorin, fibromodulin, and lumican; or
d) said enzyme is Lysyl oxidiase; or
e) said MMP is selected from the group consisting of MMP2, MMP3, MMP7, MMP9, MMP-11, MMP12, MMP13, MMP9 and MMP20; or
f) said TIMP is selected from the group consisting of TIMP1, TIMP2, TIMP3 and TIMP4.

7. The screening method according to claim 1, wherein said compound modulates said response by reducing or inhibiting RNA expression or protein expression within said skin cell.

8. A method of screening for a compound for use as a treatment of a skin condition, the method comprising:
a) obtaining skin cells from an individual comprising a skin condition in need of treatment;
b) measuring the presence of a plurality of biomolecules associated with the extracelluar matrix (ECM) from a first portion of said skin cells;
b) exposing a second portion of said skin cells to a compound suspected of providing a desired therapeutic effect associated with the extracellular matrix (ECM);
c) measuring the presence of said plurality of biomolecules in said second portion of skin cells after exposure;
d) comparing the measurements obtained from said first portion and said second portion; and
e) approving said compound for treatment of said skin condition if the comparison demonstrates a desired difference in the presence of said plurality of biomolecules.

9. The method according to claim 8, wherein said cell is selected from the group consisting of an epidermal cell, a keratinocyte, a Langerhans cell, a melanocyte, and a fibroblast.

10. The method according to claim 8, wherein said skin condition is aged skin or wrinkled skin.

11. The method according to claim 8, wherein said plurality of biomolecules are measured by a method selected from the group consisting of microarray hybridization, electrophoresis, capillary electrophoresis, liquid chromatography, reverse transcription polymerase chain reaction (RT-PCR), Enzyme-Linked ImmunoSorbent Assay (ELISA), Western Blot and Northern Blot.

12. The method according to claim 8, wherein said plurality of biomolecules are selected from the group consisting of a collagen, a fibrillin, an elastin, a fibronectin, a proteoglycan, an enzyme, a matrix metallopeptidase (MMP), a metalloproteinase inhibitor (TIMP), and a combination thereof.

13. The screening method according to claim 11, wherein:
a) said collagen is selected from the group consisting of COL1A1, COL1A2, COL2A1, COL3A1, COL4A1, COL4A2, COL4A3, COL4A4, COL5A1, COL5A2, and COL7A1; or
b) said fibrillin is Fibrillin-1 or Fibrillin-2; or
c) said proteoglycan is selected from the group consisting of aggrecan, decorin, fibromodulin, and lumican; or
d) said enzyme is Lysyl oxidiase; or
e) said MMP is selected from the group consisting of MMP2, MMP3, MMP7, MMP9, MMP-11, MMP12, MMP13, MMP9 and MMP20; or
f) said TIMP is selected from the group consisting of TIMP1, TIMP2, TIMP3 and TIMP4.

14. The method according to claim 8, wherein said desired therapeutic effect comprises increasing within skin cells, expression of a collagen and an elastin.

15. A method of validating allegations of a skin treatment product comprising:
a) contacting a skin cell with a cosmetic or therapeutic product that is alleged to treat a skin condition associated with the extracellular matrix;
b) measuring the presence of a plurality of biomolecules associated with the extracellular matrix before and after contact;
c) comparing the measurements to the allegations; and
d) confirming or deny the allegations.

16. A composition of matter for determining a profile of gene expression associated with extracellular matrix comprising a substrate comprising a plurality of biomolecules associated with the extracellular matrix (ECM) attached thereto, said plurality of biomolecules comprising nucleic acid sequences encoding a collagen, an elastin, a proteoglycan, and a metalloproteinase inhibitor (TIMP).

17. The composition of matter according to claim 15, wherein:
a) said collagen is selected from the group consisting of COL1A1, COL1A2, COL2A1, COL3A1, COL4A1, COL4A2, COL4A3, COL4A4, COL5A1, COL5A2, and COL7A1;
b) said proteoglycan is selected from the group consisting of aggrecan, decorin, fibromodulin, and lumican; and
c) said TIMP is selected from the group consisting of TIMP1, TIMP2, TIMP3 and TIMP4.

18. The composition of matter according to claim 16, further comprising nucleic acid sequences encoding:
d) Fibrillin-1 or Fibrillin-2;
e) Lysyl oxidiase; and
f) a MMP selected from the group consisting of MMP2, MMP3, MMP7, MMP9, MMP-11, MMP12, MMP13, MMP9 and MMP20.


SEQUENCE LISTING (70 sequences)

SEQ ID NO: 1 - DNA, 19 bp, Artificial
  COL1A1 primer sense
gccgtgacctcaagatgtg

SEQ ID NO: 2 - DNA, 19 bp, Artificial
  COL1A1 primer antisense
gccgaaccagacatgcctc

SEQ ID NO: 3 - DNA, 21 bp, Artificial
  COL1A2 primer sense
aattggagctgttggtaacgc

SEQ ID NO: 4 - DNA, 19 bp, Artificial
  COL1A2 primer antisense
caccagtaaggccgtttgc

SEQ ID NO: 5 - DNA, 19 bp, Artificial
  COL2A1 primer sense
acactgggactgtcctctg

SEQ ID NO: 6 - DNA, 20 bp, Artificial
  COL1A2 primer antisense
gtccaggggcacctttttca

SEQ ID NO: 7 - DNA, 19 bp, Artificial
  COL3A1 primer sense
aggtcctgcgggtaacact

SEQ ID NO: 8 - DNA, 21 bp, Artificial
  COL3A1 primer antisense
actttcacccttgacaccctg

SEQ ID NO: 9 - DNA, 20 bp, Artificial
  COL4A1 primer sense
aggggtcggagagaaaggtg

SEQ ID NO: 10 - DNA, 23 bp, Artificial
  COL4A1 primer antisense
ggtcctgtgcctataacaattcc

SEQ ID NO: 11 - DNA, 21 bp, Artificial
  COL4A2 primer sense
gagcctggattggtcggtttc

SEQ ID NO: 12 - DNA, 19 bp, Artificial
  COL4A2 primer antisense
ggtggagggtgtctgatgg

SEQ ID NO: 13 - DNA, 21 bp, Artificial
  COL4A3 primer sense
ccatagccgttcacagccaaa

SEQ ID NO: 14 - DNA, 21 bp, Artificial
  COL4A3 primer antisense
tagttgcacgttcctcttcca

SEQ ID NO: 15 - DNA, 22 bp, Artificial
  COL4A4 primer sense
ataagggtccaactggtgttcc

SEQ ID NO: 16 - DNA, 22 bp, Artificial
  COLA4 primer antisense
ctccctgaatacctttaacggc

SEQ ID NO: 17 - DNA, 21 bp, Artificial
  COL5A1 primer sense
tcaacctgtcagatggcaagt

SEQ ID NO: 18 - DNA, 19 bp, Artificial
  COL5A1 primer antisense
cggtcgaggaatttggtgg

SEQ ID NO: 19 - DNA, 21 bp, Artificial
  COL5A2 primer sense
accacagggtttacaaggaca

SEQ ID NO: 20 - DNA, 21 bp, Artificial
  COL5A2 primer antisense
tcctgcaaatcccacttcacc

SEQ ID NO: 21 - DNA, 21 bp, Artificial
  COL7A1 primer sense
gccaacctccgacttcttctt

SEQ ID NO: 22 - DNA, 22 bp, Artificial
  COL7A1 primer antisense
ctgtccactgtactctcaagga

SEQ ID NO: 23 - DNA, 20 bp, Artificial
  Elastin ELN primer sense
cgcccagtttgggttagttc

SEQ ID NO: 24 - DNA, 20 bp, Artificial
  Elastin ELN primer antisense
caccttggcagcggattttg

SEQ ID NO: 25 - DNA, 21 bp, Artificial
  FN 1 primer sense
ccccattccaggacacttctg

SEQ ID NO: 26 - DNA, 20 bp, Artificial
  FN 1 primer antisense
gcccacggtaacaacctctt

SEQ ID NO: 27 - DNA, 21 bp, Artificial
  Fibrillin-1 primer sense
caggacaggcccatgttttac

SEQ ID NO: 28 - DNA, 21 bp, Artificial
  Fibrillin-1 primer antisense
cccgtgcggatatttggaatg

SEQ ID NO: 29 - DNA, 22 bp, Artificial
  Fibrillin-2 primer sense
gtgcattgtcccgatttgtaga

SEQ ID NO: 30 - DNA, 22 bp, Artificial
  Fibrillin-2 primer antisense
cgtccaccattctgacatccat

SEQ ID NO: 31 - DNA, 21 bp, Artificial
  FBLN2 primer sense
tgtcctgtgaagacatcaacg

SEQ ID NO: 32 - DNA, 19 bp, Artificial
  FBLN2 primer antisense
gagcccttggtgttctggc

SEQ ID NO: 33 - DNA, 20 bp, Artificial
  FBLN5 primer sense
gccctactcgaacccctact

SEQ ID NO: 34 - DNA, 22 bp, Artificial
  FBLN5 primer antisense
ggagatcgtgggatagtttgga

SEQ ID NO: 35 - DNA, 23 bp, Artificial
  Lumican primer sense
tttcaatgtgtcatccctggttg

SEQ ID NO: 36 - DNA, 22 bp, Artificial
  Lumican primer antisense
ccaaacgcaaatgcttgatctt

SEQ ID NO: 37 - DNA, 22 bp, Artificial
  decorin primer sense
gtcctggagcatttacaccttt

SEQ ID NO: 38 - DNA, 21 bp, Artificial
  decorin primer antisense
tggtgcccagttctatgacaa

SEQ ID NO: 39 - DNA, 21 bp, Artificial
  fibromodulin primer sense
acttcctcacggccatgtact

SEQ ID NO: 40 - DNA, 20 bp, Artificial
  fibromodulin primer antisense
tggcattgtcaaagacgcct

SEQ ID NO: 41 - DNA, 20 bp, Artificial
  aggrecan primer sense
ctgcttccgaggcatttcag

SEQ ID NO: 42 - DNA, 20 bp, Artificial
  aggrecan primer antisense
cttgggtcacgatccactcc

SEQ ID NO: 43 - DNA, 21 bp, Artificial
  Lysyl oxidase primer sense
cgacccttacaacccctacaa

SEQ ID NO: 44 - DNA, 20 bp, Artificial
  Lysysl primer antisense
ggccagacagttttcctccg

SEQ ID NO: 45 - DNA, 21 bp, Artificial
  MMP2 primer sense
cttccaagtctggagcgatgt

SEQ ID NO: 46 - DNA, 21 bp, Artificial
  MMP2 primer antisense
taccgtcaaaggggtatccat

SEQ ID NO: 47 - DNA, 19 bp, Artificial
  MMP3 primer sense
cggttccgcctgtctcaag

SEQ ID NO: 48 - DNA, 21 bp, Artificial
  MMP3 primer antisense
cgccaaaagtgcctgtcttta

SEQ ID NO: 49 - DNA, 21 bp, Artificial
  MMP7 primer sense
ggaggagatgctcacttcgat

SEQ ID NO: 50 - DNA, 22 bp, Artificial
  MMP7 primer antisense
aggaatgtcccatacccaaaga

SEQ ID NO: 51 - DNA, 22 bp, Artificial
  MMP9 primer sense
catttcgacgatgacgagttgt

SEQ ID NO: 52 - DNA, 19 bp, Artificial
  MMP9 primer antisense
cgggtgtagagtctctcgc

SEQ ID NO: 53 - DNA, 21 bp, Artificial
  MMP-11 primer sense
tctacacctttcgctacccac

SEQ ID NO: 54 - DNA, 21 bp, Artificial
  MMP-11 primer antisense
ctccagcggtgcaatctcatt

SEQ ID NO: 55 - DNA, 21 bp, Artificial
  MMP-12 primer sense
ctcttcccctgaacagctcta

SEQ ID NO: 56 - DNA, 18 bp, Artificial
  MMP-12 primer antisense
cagttgcccggtcacttt

SEQ ID NO: 57 - DNA, 23 bp, Artificial
  MMP13 primer sense
tttcaacggacccatacagtttg

SEQ ID NO: 58 - DNA, 22 bp, Artificial
  MMP13 primer antisense
catgacgcgaacaatacggtta

SEQ ID NO: 59 - DNA, 21 bp, Artificial
  MMP19 primer sense
ccgtggactacctgtcacaat

SEQ ID NO: 60 - DNA, 22 bp, Artificial
  MMP19 primer antisense
tgagactggaagttcagatgct

SEQ ID NO: 61 - DNA, 21 bp, Artificial
  MMP20 primer sense
gagttctgtcgaggtggacaa

SEQ ID NO: 62 - DNA, 21 bp, Artificial
  MMP20 primer antisense
ccccgtgatctccattttcaa

SEQ ID NO: 63 - DNA, 19 bp, Artificial
  TIMP1 primer sense
gggttccaagccttagggg

SEQ ID NO: 64 - DNA, 22 bp, Artificial
  TIMP1 primer antisense
ttccagcaatgagaaactcctc

SEQ ID NO: 65 - DNA, 21 bp, Artificial
  TIMP2 primer sense
aagcggtcagtgagaaggaag

SEQ ID NO: 66 - DNA, 23 bp, Artificial
  TIMP2 primer antisense
ggggccgtgtagataaactctat

SEQ ID NO: 67 - DNA, 20 bp, Artificial
  TIMP3 primer sense
tgcaacttcgtggagaggtg

SEQ ID NO: 68 - DNA, 21 bp, Artificial
  TIMP3 primer antisense
cacaaagcaaggcaggtagta

SEQ ID NO: 69 - DNA, 22 bp, Artificial
  TIMP4 primer sense
ctgctacacagtaccctgtacc

SEQ ID NO: 70 - DNA, 21 bp, Artificial
  TIMP4 primer antisense
tgccgtcaacatgcttcatac